(19) United States
(12) Patent Application Publication
(10) Pub. No.: US 2014/0148548 A1
(43) Pub. Date: May 29, 2014

(54) Fluorine-Containing Polymerizable Monomer And Polymer Compound Using Same

(71) Applicant: Central Glass Company, Limited, Ube-shi (JP)

(72) Inventors: Kazuhiro YAMANAKA, Tokyo (JP); Takeshi SUDA, Fujimino-shi (JP); Hiroki UOYAMA, Iruma-gun (JP)

(21) Appl. No.: 13/868,784

(22) Filed: Apr. 23, 2013

(30) Foreign Application Priority Data
Nov. 28, 2012 (JP) 2012-260189

(51) Int. Cl. C08G 69/42; C09D 177/10; C09D 179/08; C07C 215/70

(52) U.S. Cl. CPC C08G 69/42; C07C 215/70; C09D 177/10; C09D 179/08
USPC 524/602; 564/427; 528/291

(57) ABSTRACT

The present invention provides a fluorine-containing fluorenediamine of the formula (2):

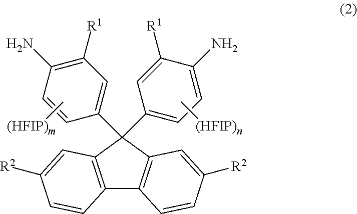

where R¹ represents one kind of substituent group selected from the group consisting of a hydrogen atom, a fluorine atom, a chlorine atom, a bromine atom, a C₁-C₄ alkyl group, a C₁-C₄ fluoroalkyl group in which any number of hydrogen atoms can be substituted by a fluorine atom, a phenyl group, a methoxy group and a nitro group; R² represents one kind of substituent group selected from the group consisting of a hydrogen atom, a fluorine atom, a chlorine atom, a bromine atom, an iodine atom, a phenyl group, a naphthyl group, a biphenyl group, a sulfonic acid group, a —C≡C—C(CH₃)₂OH group, a —C≡C—C₆H₅ group and a —C≡C—Si(CH₃)₃ group; HFIP represents a —C(CF₃)₂OH group; and m and n each independently represent an integer of 0 to 2 and satisfy a condition of 1≦m+n≦4.

### FIELD OF THE INVENTION

The present invention relates to a novel fluorene-containing polymer compound useful as materials for protective films of electronic circuit boards, protective films of semiconductor devices, gas separation membranes and the like. The present invention also relates to a fluorene-containing polymerizable monomer for the production of the fluorene-containing polymer compound.

### BACKGROUND OF THE INVENTION

Aromatic diamine compounds are useful as raw materials for heat-resistant polymers such as polyamide, polyimide and polybenzoxazole. These polymers are used in the fields of automotive vehicles, aerospace applications, fireproof suits etc. because of their high heat resistance. However, many of these polymers are low in solubility in organic solvents and low in moldability. It is thus common practice to, obtain a final molded product of e.g. polyimide or polybenzoxazole by molding an organic solvent-soluble polymer precursor (such as polyamic acid or polyamide-phenol) into a desired shape and converting the polymer precursor to polyimide or polybenzoxazole through dehydration cyclization reaction under high-temperature conditions of 300 to 350° C.

The above molding process is however not suitable for the production of relatively low heat-resistant display parts using organic dyes, semiconductor lamination parts having different thermal expansion coefficients and susceptible to residual thermal stresses and the like because the molding temperature is too high. For this reason, attention is being given to organic solvent-soluble polyimide and polybenzoxazole materials that are moldable at temperature levels where organic solvents can be vaporized and volatilized

As techniques for making the polymers soluble in organic solvents, it is known to: introduce a bent structure e.g. ether bond or methylene bond into the main chain skeletons of the polymers; introduce a bulky structure e.g. tert-butyl group or trifluoromethyl group into the side chains of the polymers; and disturb the symmetry of the main chain skeletons of the polymers. Particularly known is a technique using a fluorene structure to improve the solubility of the polymers in organic solvents without sacrificing heat resistance.

It has been reported that fluorene-containing polymers show not only solubility in organic solvents but also low dielectric constant and gas permeation improvement effect because of their specific structure and thus have uses as materials for protective films of semiconductor devices and gas separation membranes (see Patent Documents 1 and 2).

The fluorine-containing polymers however have drawbacks such as low applicability to hydrophilic substrates due to high hydrophobicity and low mechanical strength due to small intermolecular interaction. There are reported some attempts to resolve these drawbacks by the introduction of a hydrophilic group e.g. phenolic hydroxy group or carboxyl group into the fluorene structures of the polymers. The introduction of such a hydrophilic group e.g. phenolic hydroxy group or carboxyl group requires a plurality of synthesis steps and complicated operations (see Patent Documents 3, 4 and 8).

More specifically, Patent Document 3 discloses a process for production of a phenolic hydroxy group-containing fluorenediamine. In this production process, the target phenolic hydroxy group-containing fluorenediamine is obtained by introducing a phenolic hydroxy group into a fluorene structure, and then, subjecting the resulting compound to nitration and hydrogen reduction. It is described in the working examples of Patent Document 3 that the target product can be obtained with relatively high yield (85 to 90%) in each step. However, this production process is hardly said as an industrially easy technique in view of the problem of waste due to the use of a large amount of nitric acid, the need to use an expensive palladium and the need to high-pressure hydrogen.

Patent Document 4 discloses a process for production of a calboxyl group-containing florenediamine. In this production process, the target carboxyl group-containing fluorenediamine is obtained by heating diphenic acid in sulfuric acid, and then, causing dehydration of the resulting fluorenone-carboxylic acid with aniline under required high-temperature conditions. This production process is also hardly said as an industrially easy technique in view of the problem of waste due to the use of a large amount of sulfuric acid and the need to adopt additional equipment for azeotropic dehydration in the coexistence of toluene in the dehydration step.

Patent Document 8 discloses a process for production of a phenolic hydroxy group-containing fluorenediamine, which is opposite in phenolic hydroxyl group substitution position and amino group substitution position to those of Patent Document 3. In this production process, 2-benzoxazoline for general purpose is used. It is however necessary to perform purification operation for separation of the target product from by-products by silica gel chromatography. This process is thus also hardly said as an industrially easy technique.

There have accordingly been a demand to develop techniques for easy introduction of a phenolic hydroxy group, a carboxyl group or any other hydrophilic functional group into the fluorene structures.

Further, there has been known no fluorene-containing aromatic diamine compound having introduced thereto a 2-hydroxy-1,1,1,3,3,3-hexyafluoroisopropyl group.

### PRIOR ART DOCUMENTS

Patent Document 1: Japanese Laid-Open Patent Publication No. H11-343414
Patent Document 2: Japanese Laid-Open Patent Publication No. S63-190607
Patent Document 3: Japanese Laid-Open Patent Publication No. 2002-105034
Patent Document 4: Japanese Laid-Open Patent Publication No. H05-194338
Patent Document 5: Japanese Laid-Open Patent Publication No. 2007-119503
Patent Document 6: Japanese Laid-Open Patent Publication No. 2007-119504
Patent Document 7: Japanese Laid-Open Patent Publication No. 2008-150534
Patent Document 8: Japanese Laid-Open Patent Publication No. 2009-235048

### SUMMARY OF THE INVENTION

It is therefore an object of the present invention to provide a fluorene-containing aromatic diamine compound having introduced thereto a 2-hydroxy-1,1,1,3,3,3-hexyafluoroisopropyl group (hereinafter sometimes referred to as “HFIP group” or “—C(CF₃)₂OH group”), which is called a “HFIP-containing fluorenediamine”. It is also an object of the present invention to provide a polyamide compound, a polyimide compound and a polybenzoxazole-analogous fluorine-containing heterocyclic polymer compound, each of which is produced from the HFIP-containing fluorenediamine.

As a result of extensive researches made to achieve the above objects, the present inventors have found a easier synthesis method for introduction of a hydrophilic HFIP group into a fluorene structure than conventional methods for introduction of a hydrophilic group e.g. phenolic hydroxy group or carboxyl group into a fluorene structure, and then, have produced a HFIP-containing fluorenediamine by such a method. The present inventors have further produced a polyamide compound, a polyimide compound and a polybenzoxazole-analogous fluorine-containing heterocyclic polymer compound from the HFIP-containing fluornediamine. The present invention was accomplished based on the above findings.

Namely, the present invention includes the following aspects.

[Inventive Aspect 1]

A fluorine-containing fluorenediamine of the formula (2):

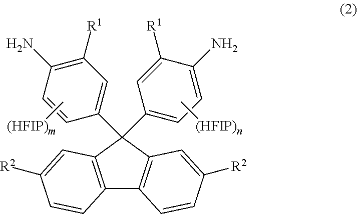

where R¹ represents one kind of substituent group selected from the group consisting of a hydrogen atom, a fluorine atom, a chlorine atom, a bromine atom, a C₁-C₄ alkyl group, a C₁-C₄ fluoroalkyl group in which any number of hydrogen atoms can be substituted by a fluorine atom, a phenyl group, a methoxy group and a nitro group; R² represents one kind of substituent group selected from the group consisting of a hydrogen atom, a fluorine atom, a chlorine atom, a bromine atom, an iodine atom, a phenyl group, a naphthyl group, a biphenyl group, a sulfonic acid group, a —C≡C—C(CH₃)₂OH group, a —C≡C—C₆H₅ group and a —C≡C—Si(CH₃)₃ group; HFIP represents a —C(CF₃)₂OH group; and m and n each independently represent an integer of 0 to 2 and satisfy a condition of 1≦m+n≦4.

[Inventive Aspect 2]

The fluorine-containing fluorenediamine according to Inventive Aspect 1, wherein the fluorine-containing fluorenediamine is of the formula (3-1):

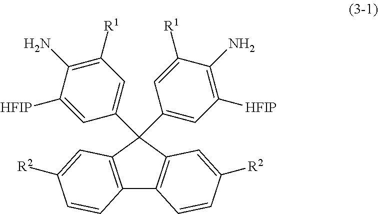

where R¹, R² and HFIP have the same meanings as those in the formula (2).

[Inventive Aspect 3]

The fluorine-containing fluorenediamine according to Inventive Aspect 1 or 2, wherein the fluorine-containing fluorenediamine is of the formula (2B):

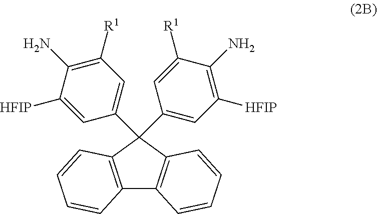

where R¹ and HFIP have the same meanings as those in the formula (2).

[Inventive Aspect 4]

A polymer compound obtained by reaction of the fluorine-containing fluorenediamine according to any one of Inventive Aspects 1 to 3 with a dicarboxylic acid or dicarboxylic acid derivative of the formula (10) or (11) or a tetracarboxylic acid dianhydride of the formula (14)

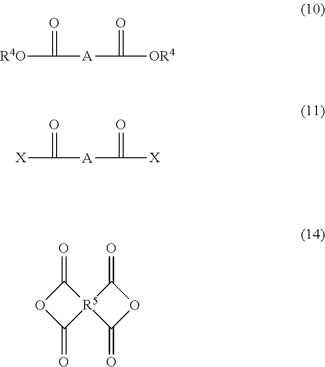

where A represents a divalent organic group having one or more kinds selected from the group consisting of alicyclic, aromatic and alkylene groups and may contain an oxygen atom, a sulfur atom or a nitrogen atom; any number of hydrogen atoms in A may be substituted by an alkyl group, a fluorine atom, a chlorine atom, a fluoroalkyl group, a carboxyl group, a hydroxy group or a cyano group; R⁴ each independently represents a hydrogen atom, a C₁-C₁₀ alkyl group or a benzyl group; X represents a fluorine atom, a chlorine atom, a bromine atom or an iodine atom; R⁵ represents a tetravalent organic group having one or more kinds selected from the group consisting of alicyclic, aromatic and alkylene groups and may contain a fluorine atom, a chlorine atom, an oxygen atom, a sulfur atom or a nitrogen atom; and any number of hydrogen atoms in R⁵ may be substituted by an alkyl group, a fluoroalkyl group, a carboxyl group, a hydroxy group or a cyano group.

[Inventive Aspect 5]

A polymer compound comprising at least a repeating unit of the formula (6):

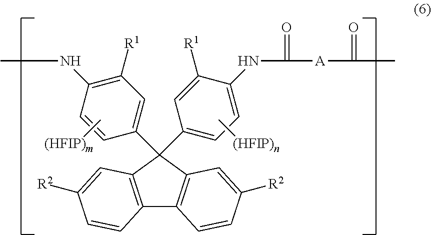

where R¹ represents one kind of substituent group selected from the group consisting of a hydrogen atom, a fluorine atom, a chlorine atom, a bromine atom, a C₁-C₄ alkyl group, a C₁-C₄ fluoroalkyl group in which any number of hydrogen atoms can be substituted by a fluorine atom, a phenyl group, a methoxy group and a nitro group; R² represents one kind of substituent group selected from the group consisting of a hydrogen atom, a fluorine atom, a chlorine atom, a bromine atom, an iodine atom, a phenyl group, a naphthyl group, a biphenyl group, a sulfonic acid group, a —C≡C—C(CH₃)₂OH group, a —C≡C—C₆H₅ group and a —C≡C—Si(CH₃)₃ group; A represents a divalent organic group having one or more kinds selected from the group consisting of alicyclic, aromatic and alkylene groups and may contain an oxygen atom, a sulfur atom or a nitrogen atom; any number of hydrogen atoms in A may be substituted by an alkyl group, a fluorine atom, a chlorine atom, a fluoroalkyl group, a carboxyl group, a hydroxy group or a cyano group; HFIP represents a —C(CF₃)₂OH group; and m and n each independently represent an integer of 0 to 2 and satisfy a condition of 1≦m+n≦4.

[Inventive Aspect 6]

The polymer compound according to Inventive Aspect 5, wherein the repeating unit is of the formula (19):

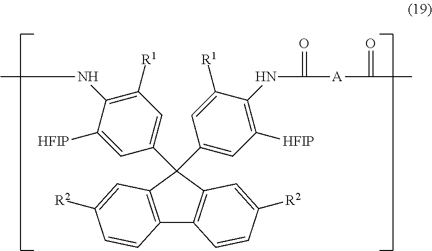

where R¹, R², A and HFIP have the same meanings as those in the formula (6).

[Inventive Aspect 7]

The polymer compound according to Inventive Aspect 5 or 6, wherein the repeating unit is of the formula (6A):

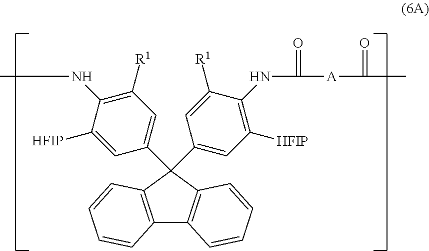

where R¹, A and HFIP have the same meanings as those in the formula (6).

[Inventive Aspect 8]

A polymer compound comprising at least a repeating unit of the formula (9):

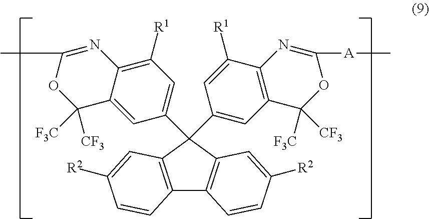

where R¹ represents one kind of substituent group selected from the group consisting of a hydrogen atom, a fluorine atom, a chlorine atom, a bromine atom, a C₁-C₄ alkyl group, a C₁-C₄ fluoroalkyl group in which any number of hydrogen atoms can be substituted by a fluorine atom, a phenyl group, a methoxy group and a nitro group; R² represents one kind of substituent group selected from the group consisting of a hydrogen atom, a fluorine atom, a chlorine atom, a bromine atom, an iodine atom, a phenyl group, a naphthyl group, a biphenyl group, a sulfonic acid group, a —C≡C—C(CH₃)₂OH group, a —C≡C—C₆H₅ group and a —C≡C—Si(CH₃)₃ group; A represents a divalent organic group having one or more kinds selected from the group consisting of alicyclic, aromatic and alkylene groups and may contain an oxygen atom, a sulfur atom or a nitrogen atom; and any number of hydrogen atoms in A may be substituted by an alkyl group, a fluorine atom, a chlorine atom, a fluoroalkyl group, a carboxyl group, a hydroxy group or a cyano group.

[Inventive Aspect 9]

The polymer compound according to Inventive Aspect 8, wherein the repeating unit is of the formula (9B):

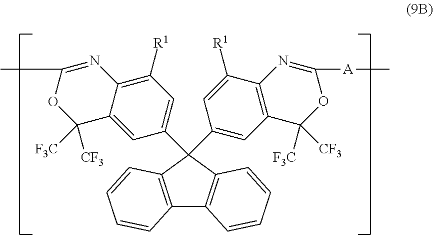

where R¹ and A have the same meanings as those in the formula (9).

[Inventive Aspect 10]

A polymer compound comprising at least a repeating unit of the formula (7):

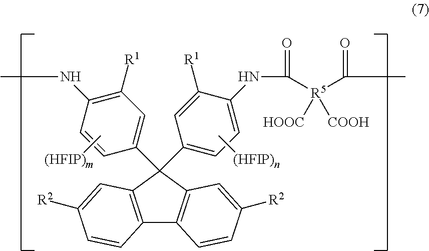

where R¹ represents one kind of substituent group selected from the group consisting of a hydrogen atom, a fluorine atom, a chlorine atom, a bromine atom, a C₁-C₄ alkyl group, a C₁-C₄ fluoroalkyl group in which any number of hydrogen atoms can be substituted by a fluorine atom, a phenyl group, a methoxy group and a nitro group; R² represents one kind of substituent group selected from the group consisting of a hydrogen atom, a fluorine atom, a chlorine atom, a bromine atom, an iodine atom, a phenyl group, a naphthyl group, a biphenyl group, a sulfonic acid group, a —C≡C—C(CH₃)₂OH group, a —C≡C—C₆H₅ group and a —C≡C—Si(CH₃)₃ group; R⁵ represents a tetravalent organic group having one or more kinds selected from the group consisting of alicyclic, aromatic and alkylene groups and may contain a fluorine atom, a chlorine atom, an oxygen atom, a sulfur atom or a nitrogen atom; and any number of hydrogen atoms in R⁵ may be substituted by an alkyl group, a fluoroalkyl group, a carboxyl group, a hydroxy group or a cyano group; HFIP represents a —C(CF₃)₂OH group; and m and n each independently represent an integer of 0 to 2 and satisfy a condition of 1≦m+n≦4.

[Inventive Aspect 11]

The polymer compound according to Inventive Aspect 10, wherein the repeating unit is of the formula (7A):

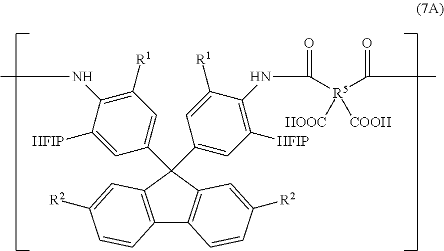

where R¹, R², R⁵ and HFIP have the same meanings as those in the formula (7).

[Inventive Aspect 12]

The polymer compound according to Inventive Aspect 10 or 11, wherein the repeating unit is of the formula (7B):

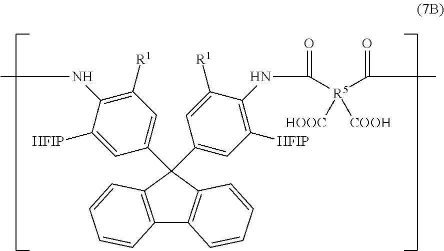

where R¹, R⁵ and HFIP have the same meanings as those in the formula (7).

[Inventive Aspect 13]

A polymer compound comprising at least a repeating unit of the formula (8):

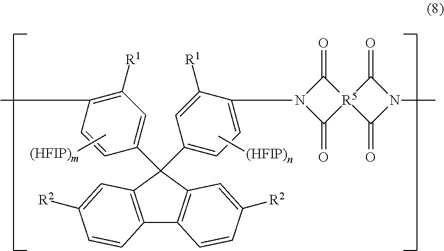

where R¹ represents one kind of substituent group selected from the group consisting of a hydrogen atom, a fluorine atom, a chlorine atom, a bromine atom, a C₁-C₄ alkyl group, a C₁-C₄ fluoroalkyl group in which any number of hydrogen atoms can be substituted by a fluorine atom, a phenyl group, a methoxy group and a nitro group; R² represents one kind of substituent group selected from the group consisting of a hydrogen atom, a fluorine atom, a chlorine atom, a bromine atom, an iodine atom, a phenyl group, a naphthyl group, a biphenyl group, a sulfonic acid group, a —C≡C—C(CH₃)₂OH group, a —C≡C—C₆H₅ group and a —C≡C—Si(CH₃)₃ group; R⁵ represents a tetravalent organic group having one or more kinds selected from the group consisting of alicyclic, aromatic and alkylene groups and may contain a fluorine atom, a chlorine atom, an oxygen atom, a sulfur atom or a nitrogen atom; and any number of hydrogen atoms in R⁵ may be substituted by an alkyl group, a fluoroalkyl group, a carboxyl group, a hydroxy group or a cyano group; HFIP represents a —C(CF₃)₂OH group; and m and n each independently represent an integer of 0 to 2 and satisfy a condition of 1≦m+n≦4.

[Inventive Aspect 14]

The polymer compound according to Inventive Aspect 13, wherein the repeating unit is of the formula (8A):

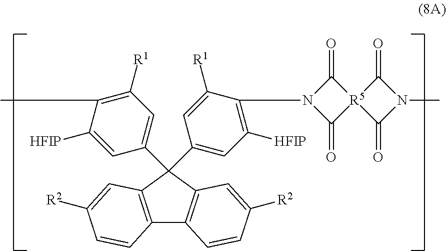

where R¹, R², R⁵ and HFIP have the same meanings as those in the formula (8).

[Inventive Aspect 15]

The polymer compound according to Inventive Aspect 13 or 14, wherein the repeating unit is of the formula (8B):

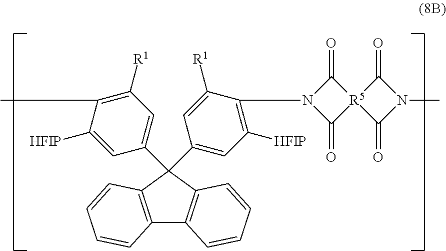

where R¹, R⁵ and HFIP have the same meanings as those in the formula (8).

[Inventive Aspect 16]

The polymer compound according to any one of Inventive Aspects 5 to 9, wherein the divalent group A is one or more kinds selected from the group consisting of groups of the formulas (25) to (29):

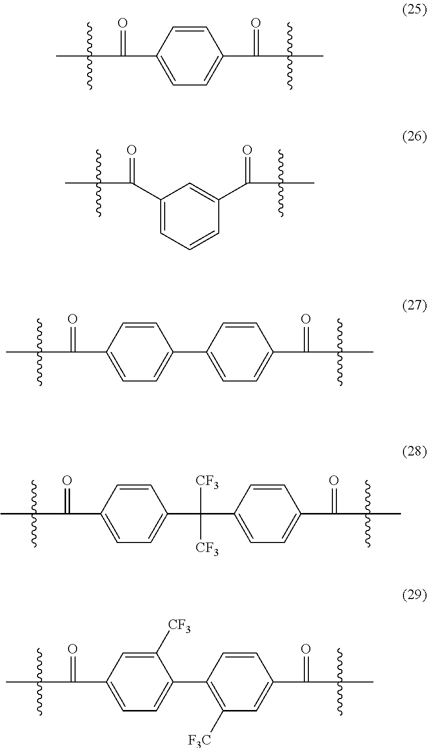 [SMILES: *C(=O)c1ccc(C(*)=O)cc1 ; *C(=O)c1cccc(C(*)=O)c1 ; *C(=O)c1ccc(-c2ccc(C(*)=O)cc2)cc1 ; *C(=O)c1ccc(C(c2ccc(C(*)=O)cc2)(C(F)(F)F)C(F)(F)F)cc1 ; *C(=O)c1ccc(-c2ccc(C(*)=O)cc2C(F)(F)F)c(C(F)(F)F)c1]

wherein each line intersecting a wavy line represents a bonding position.

[Inventive Aspect 17]

The polymer compound according to any one of Inventive Aspects 10 to 15, wherein R⁵ is one or more kinds selected from the group consisting of groups of the formulas (30) to (35):

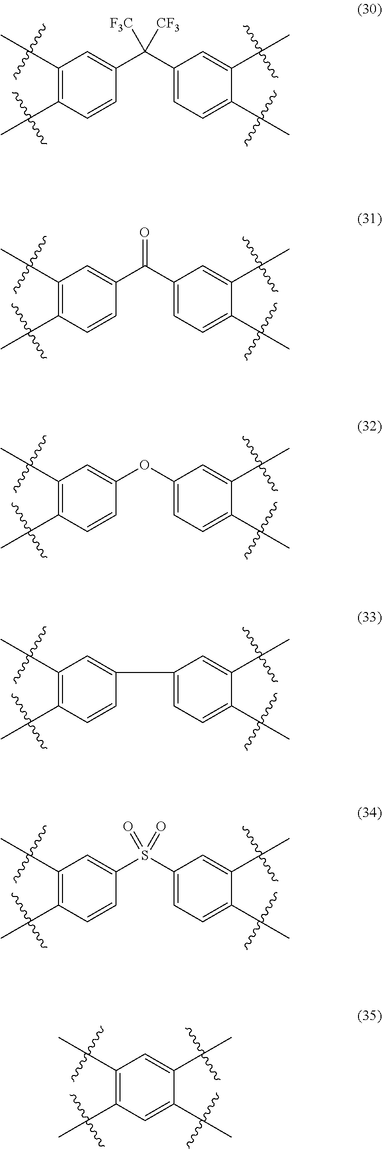 [SMILES: *c1ccc(C(c2ccc(*)c(*)c2)(C(F)(F)F)C(F)(F)F)cc1* ; *c1ccc(C(=O)c2ccc(*)c(*)c2)cc1* ; *c1ccc(Oc2ccc(*)c(*)c2)cc1* ; *c1ccc(-c2ccc(*)c(*)c2)cc1* ; *c1ccc(S(=O)(=O)c2ccc(*)c(*)c2)cc1* ; *c1cc(*)c(*)cc1*]

wherein each line intersecting a wavy line represents a bonding position.

It is possible according to the present invention to produce the HFIP-containing fluorenediamine. It is also possible according to the present invention to produce the polyamide compound, the polyimide compound and the polybenzoxazole-analogous fluorine-containing heterocyclic polymer compound from the HFIP-containing fluorenediamine.

### DESCRIPTION OF THE EMBODIMENTS

Hereinafter, the present invention will be described below. It is noted that the present invention is not limited to the following embodiments.

Among aromatic diamine compounds having a fluorene structure, 9,9-bis(4-aminophenyl)fluorene of the following formula (1) is referred to as “fluorenediamine” in the present specification. The fluorenediamine may have a substituent group.

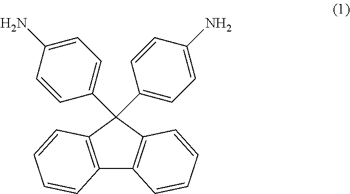 [SMILES: Nc1ccc(C2(c3ccc(N)cc3)c3ccccc3-c3ccccc32)cc1]

In the following description, a fluorenediamine having a HFIP group as a substituent group is referred to as a HFIP-containing fluorenediamine for the purposes of distinction.

A HFIP-containing fluorenediamine according to the present invention as well as a polyamide compound, a polyimide group and a polybenzoxazole-analogous fluorine-containing heterocyclic polymer compound, each of which is produced by polymerization of the HFIP-containing fluorenediamine as a monomer, will be explained below one by one.

[HFIP-Containing Fluorenediamine]

The HFIP-containing fluorenediamine according to the present invention is expressed by the formula (2).

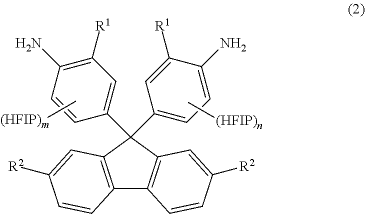

In the formula (2), R¹ represents one kind of substituent group selected from the group consisting of a hydrogen atom, a fluorine atom, a chlorine atom, a bromine atom, a C₁-C₄ alkyl group, a C₁-C₄ fluoroalkyl group in which any number of hydrogen atoms can be substituted by a fluorine atom, a phenyl group, a methoxy group and a nitro group; R² represents one kind of substituent group selected from the group consisting of a hydrogen atom, a fluorine atom, a chlorine atom, a bromine atom, an iodine atom, a phenyl group, a naphthyl group, a biphenyl group, a sulfonic acid group, a —C≡C—C(CH₃)₂OH group, a —C≡C—C₆H₅ group and a —C≡C—Si(CH₃)₃ group; HFIP represents a —C(CF₃)₂OH group; and m and n each independently represent an integer of 0 to 2 and satisfy a condition of 1≦m+n≦4.

Among the HFIP-containing fluorenediamine of the formula (2) (hereinafter sometimes referred to as “HFIP-containing fluorenediamine (2)”), preferred are those of the formulas (3-1) and (3-2).

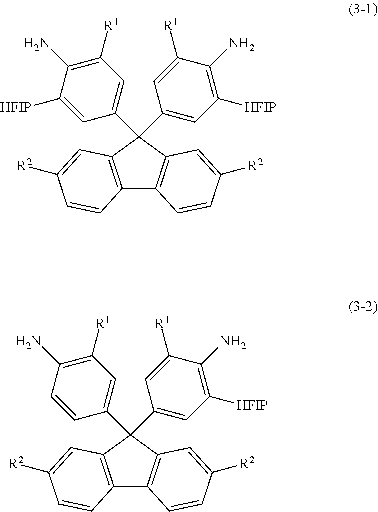

In the formulas (3-1) and (3-2), R¹, R² and HFIP have the same meanings as those in the formula (2)

Examples of the C₁-C₄ alkyl group are methyl, ethyl, propyl, isopropyl, n-butyl, sec-butyl and tert-butyl.

Examples of the C₁-C₄ fluoroalkyl group in which any number of hydrogen atoms can be substituted by a fluorine atom are trifluoromethyl, perfluoroethyl, perfluoropropyl, perfluoroisopropyl, perfluorobutyl, perfluoro-sec-butyl and perfluoro-tert-butyl.

Specific examples of the HFIP-containing fluorenediamine are those of the following formulas (4-1) to (4-12).

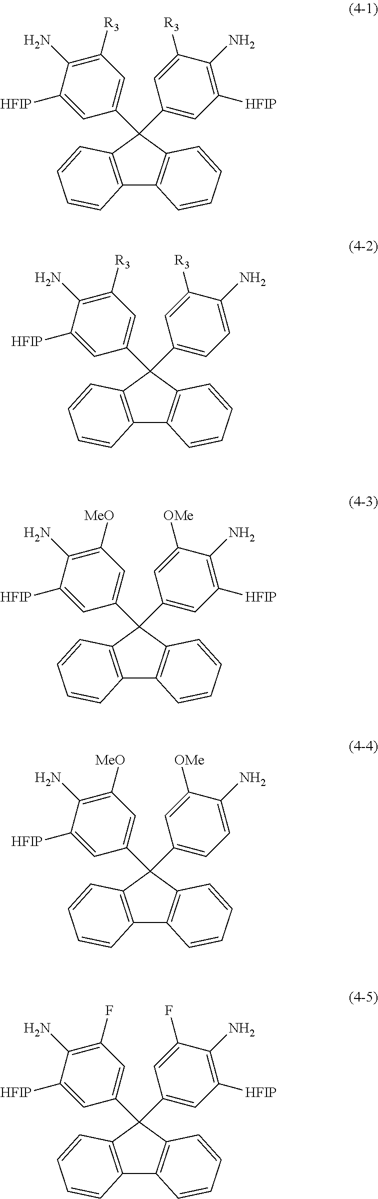

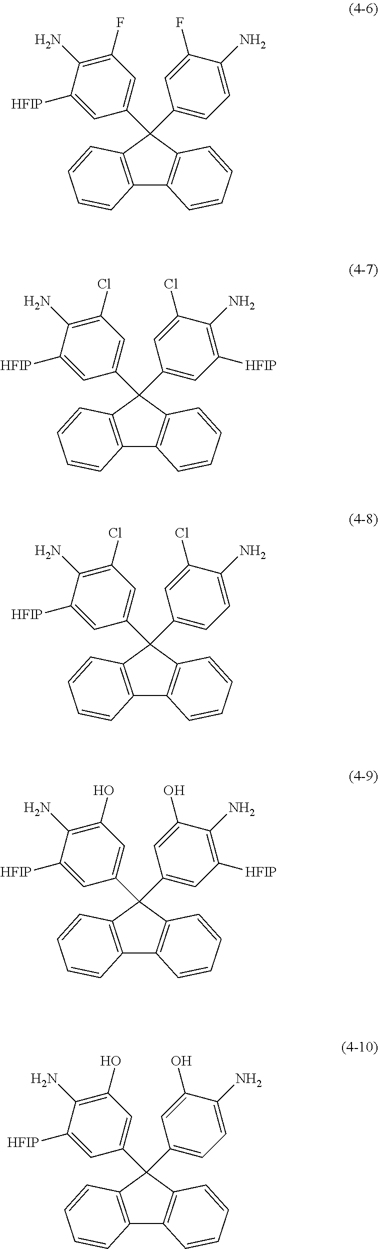

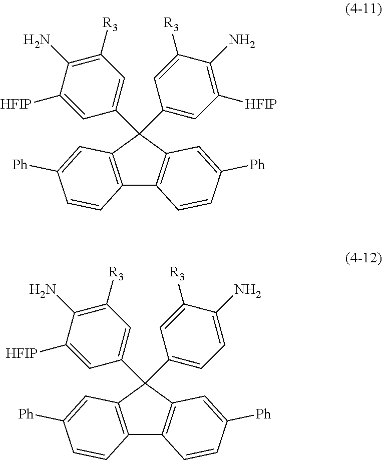

In the formulas (4-1) to (4-12), R³ represents a hydrogen atom or a C₁-C₄ alkyl group; Me represents a methyl group; Ph represents a phenyl group; and HFIP has the same meaning as in the formula (2).
Among others, the HFIP-containing fluorenediamines of the formulas (4-1) and (4-2) are particularly preferred in view of the availability of fluorenediamines as the raw material for the synthesis of the HFIP-containing fluorenediamine.

In the present invention, it is feasible to synthesize the HFIP-containing fluorenediamine (2) by reaction of a fluorenediamine of the formula (5) (sometimes referred to as “fluorenediamine (5)”) with hexafluoroacetone or hexafluoroacetone trihydrate (see Patent Documents 5, 6 and 7).

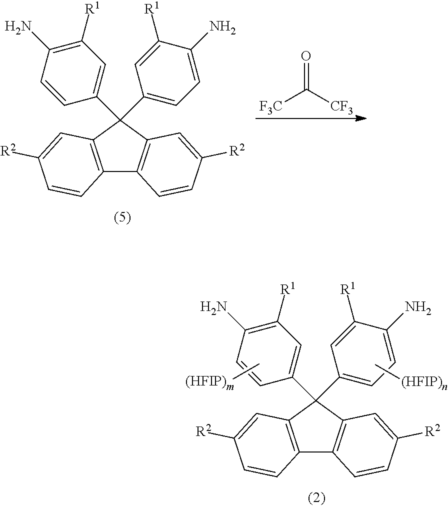

In the formula (5), R¹ and R² have the same meanings as those in the formula (2).

The reaction can be performed with or without the use of a solvent. There is no particular limitation on the kind of the solvent used as long as the solvent is not involved in the reaction. Suitable examples of the solvent are: aromatic hydrocarbons such as xylene, toluene, benzene, anisole, diphenyl ether, nitrobenzene and benzonitrile; and water. There is also no particular limitation on the amount of the solvent used. It is however unfavorable to use the solvent in a large amount because the use of a large amount of solvent leads to deterioration in yield per volume.

There is no particular limitation on the reactor used in the reaction. In the case of a sealed reactor (autoclave), the procedure of the reaction varies depending on whether to use hexafluoroacetone or hexafluoroacetone trihydrate. When hexafluoroacetone is used as the reaction substrate, it is desirable to first place the fluorenediamine (5) and, optionally, the catalyst and/or the solvent into the reactor and then introduce the hexafluoroacetone gradually into the reactor, while raising the temperature, in such a manner that the inner pressure of the reactor does not exceed 0.5 MPa.

When hexafluoroacetone trihydrate is used as the reaction substrate, the reaction can be performed by first placing the fluorenediamine (5) together with a required amount of hexafluoroacetone trihydrate into the reactor and then optionally placing the catalyst and/or the solvent into the reactor.

Further, there is no particular limitation on the reaction time of the reaction. The optimum reaction time varies depending on the temperature, the amount of the catalyst used and the like. It is thus desirable to perform the reaction, while monitoring the progress of the reaction by any ordinary analytical means such as gas chromatography, and complete the reaction after confirming that the raw substrate material has sufficiently been consumed. After the completion of the reaction, the HFIP-containing fluorenediamine (2) can be obtained by any ordinary means such as extraction, distillation or crystallization. The HFIP-containing fluorenediamine (2) can be purified by column chloromatography, recrystallization or the like as needed.

[Polymer Compound]

By polymerization of the HFIP-containing fluorenediamine (2) as the monomer, there are obtained a polyamide compound having at least a repeating unit of the formula (6) (sometimes referred to as “polyamide compound (6)”), a polyamide compound having at least a repeating unit of the formula (7) (sometimes referred to as “polyamide compound (7)”) and a polyimide compound having at least a repeating unit of the formula (8) (sometimes referred to as “polyimide compound (8)”)

These polymer compounds will be explained below one by one. A polybenzoxazole-analogous fluorine-containing heterocyclic polymer compound having at least a repeating unit of the formula (9) (sometimes referred to as “fluorine-containing heterocyclic polymer compound (9)”) as a polymerization product of the HFIP-containing fluorenediamine (2) will also be explained below.

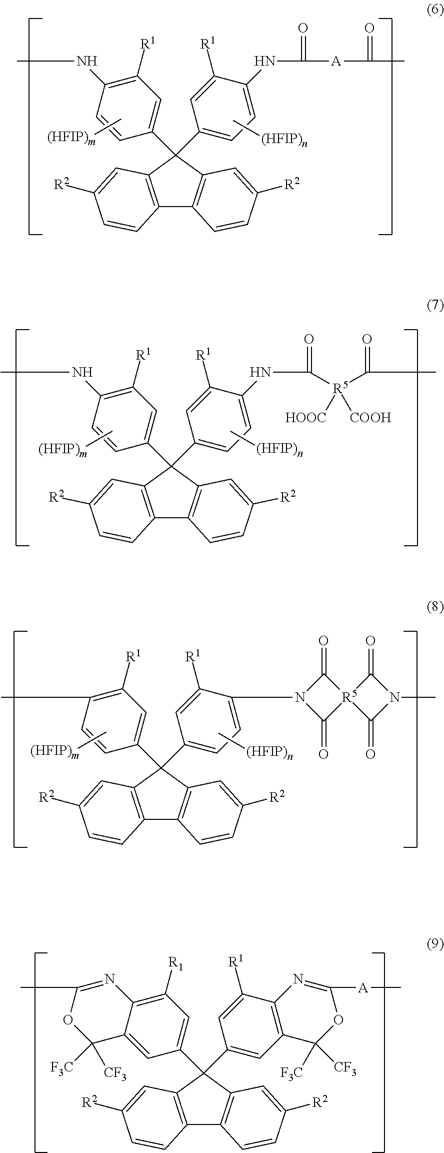

[Polyamide compound (6)]

As a polymer of the HFIP-containing fluorenediamine according to the present invention, the polyamide compound (6) is synthesized by reaction of the HFIP-containing fluorenediamine (2) with a carboxylic acid or carboxylic acid derivative of the formula (10) or (11).

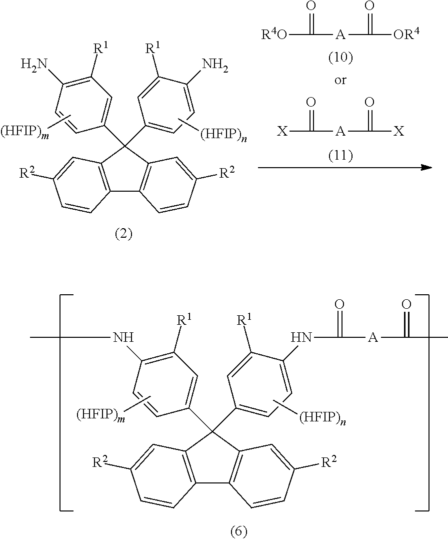

In the above formulas, R¹, R², HFIP, m and n have the same meanings as those in the formula (2); R⁴ each independently represents a hydrogen atom, a C₁-C₁₀ alkyl group or a benzyl group; A represents a divalent organic group having one or more kinds selected from the group consisting of alicyclic, aromatic and alkylene groups and may contain an oxygen atom, a sulfur atom or a nitrogen atom; any number of hydrogen atoms in A may be substituted by an alkyl group, a fluorine atom, a chlorine atom, a fluoroalkyl group, a carboxyl group, a hydroxy group or a cyano group; and X represents a fluorine atom, a chlorine atom, a bromine atom or an iodine atom.

As the dicarboxylic acid or dicarboxylic acid derivative of the formula (10) or (11), there can be used any of aliphatic dicarboxylic acids, aromatic dicarboxylic acids and derivatives thereof.

Examples of the aliphatic dicarboxylic acids and derivatives thereof are: dicarboxylic acid compounds such as oxalic acid, malonic acid, succinic acid, gultaric acid, adipic acid, pimelic acid, suberic acid, azelaic acid and sebacic acid; and derivatives thereof.

Examples of the aromatic dicarboxylic acids and derivatives thereof are phthalic acid, isophthalic acid, terephthalic acid, 4,4′-dicarboxybiphenyl, 3,3′-dicarboxyldiphenyl ether, 3,4′-dicarboxyldiphenyl ether, 4,4′-dicarboxyldiphenyl ether, 3,3′-dicarboxyldiphenylmethane, 3,4′-dicarboxyldiphenylmethane, 4,4′-dicarboxyldiphenylmethane, 3,3′-dicarboxyldiphenyldifluoromethane, 3,4′-dicarboxyldiphenyldifluoromethane, 4,4′-dicarboxyldiphenyldifluoromethane, 3,3′-dicarboxyldiphenylsulfone, 3,4′-dicarboxyldiphenylsulfone, 4,4′-dicarboxyldiphenylsulfone, 3,3′-dicarboxyldiphenyl sulfide, 3,4′-dicarboxyldiphenyl sulfide, 4,4′-dicarboxyldiphenyl sulfide, 3,3′-dicarboxyldiphenyl ketone, 3,4′-dicarboxyldiphenyl ketone, 4,4′-dicarboxyldiphenyl ketone, 2,2′-ditrifluoromethyl-4,4′-dicarboxybiphenyl, 2,2-bis(3-carboxyphenyl)propane, 2,2-bis(3,4′-dicarboxyphenyl)propane, 2,2-bis(4-carboxyphenyl)propane, 2,2-bis(3-carboxyphenyl)hexafluoropropane, 2,2-bis(3,4′-dicarboxyphenyl)hexafluoropropane, 2,2-bis(4-carboxyphenyl)hexafluoropropane, 1,3-bis(3-carboxyphenoxy)benzene, 1,4-bis(3-carboxyphenoxy)benzene, 1,4-bis(4-carboxyphenoxy)benzene, 3,3′-(1,4-phenylenebis(1-methylethylidene))bisbenzoic acid, 3,4′-(1,4-phenylenebis(1-methylethylidene))bisbenzoic acid, 4,4′-(1,4-phenylenebis(1-methylethylidene))bisbenzoic acid, 2,2-bis(4-(3-carboxyphenoxy)phenyl)propane, 2,2-bis(4-(4-carboxyphenoxy)phenyl)propane, 2,2-bis(4-(3-carboxyphenoxy)phenyl)hexafluoropropane, 2,2-bis(4-(4-carboxyphenoxy)phenyl)hexafluoropropane, bis(4-(3-carboxyphenoxy)phenyl)sulfide, bis(4-(4-carboxyphenoxy)phenyl)sulfide, bis(4-(3-carboxyphenoxy)phenyl)sulfone, bis(4-(4-carboxyphenoxy)phenyl)sulfone, perfluorononenyloxy-containing dicarboxylic acids such as 5-(perfluorononenyloxy)isophthalic acid and 4-(perfluorononenyloxy)phthalic acid, and derivatives thereof. Further, there can be used 2-(perfluorononenyloxy)terephthalic acid, 4-methoxy-5-(perfluorononenyloxy)isophthalic acid and derivatives thereof. There can also be used perfluorohexenyloxy-containing dicarboxylic acids such as 5-(perfluorohexenyloxy)isophthalic acid, 4-(perfluorohexenyloxy)phthalic acid, 2-(perfluorohexenyloxy)terephthalic acid and 4-methoxy-5-(perfluorohexenyloxy)isophthalic acid and derivatives thereof.

Among others, preferred are terephthalic acid, isophthalic acid, 4,4′-dicarboxybiphenyl, 2,2′-ditrifluoromethyl-4,4′-dicarboxybiphenyl and 2,2-bis(4-carboxyphenyl)hexafluoropropane in view of its availability and ease of condensation polymerization and in view of the transparency of the polymerization product.

The polyamide compound (6) is preferably a polyamide compound having at least a repeating unit of either one of the following formulas (12-1) to (12-5).

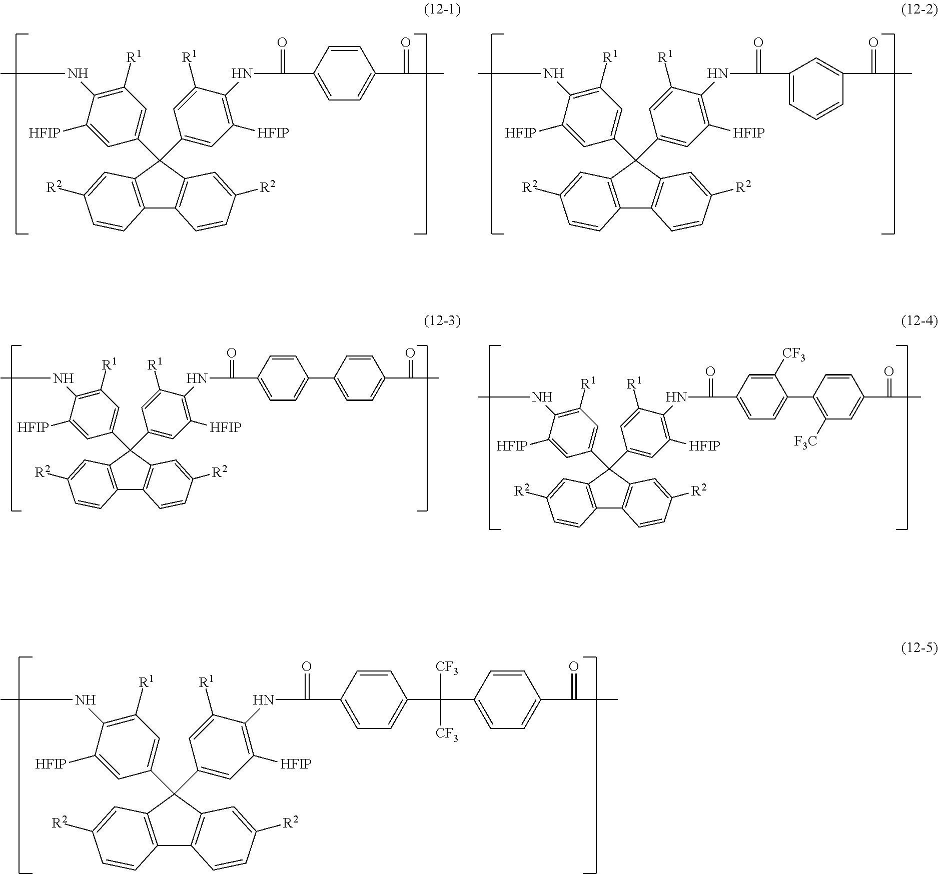

In the formulas (12-1) to (12-5), R¹, R² and HFIP have the same meanings as those in the formula (2).
Specific examples of the polyamide compound are those each having at least a repeating unit of either one of the following formulas (13-1) to (13-10).

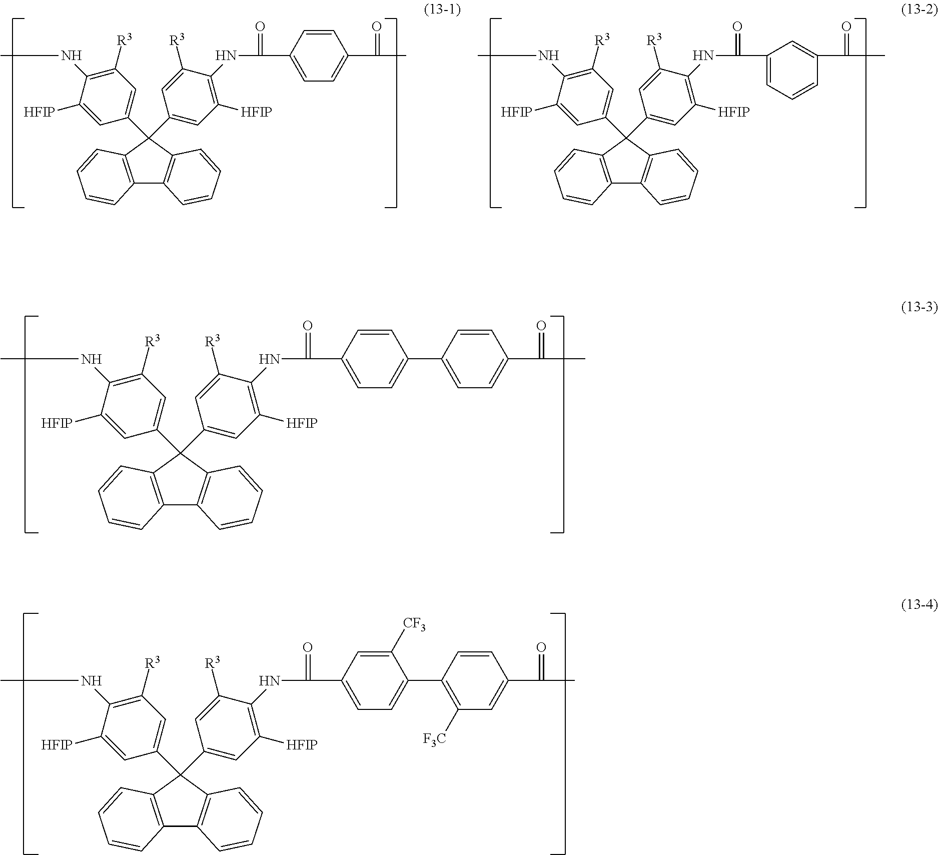

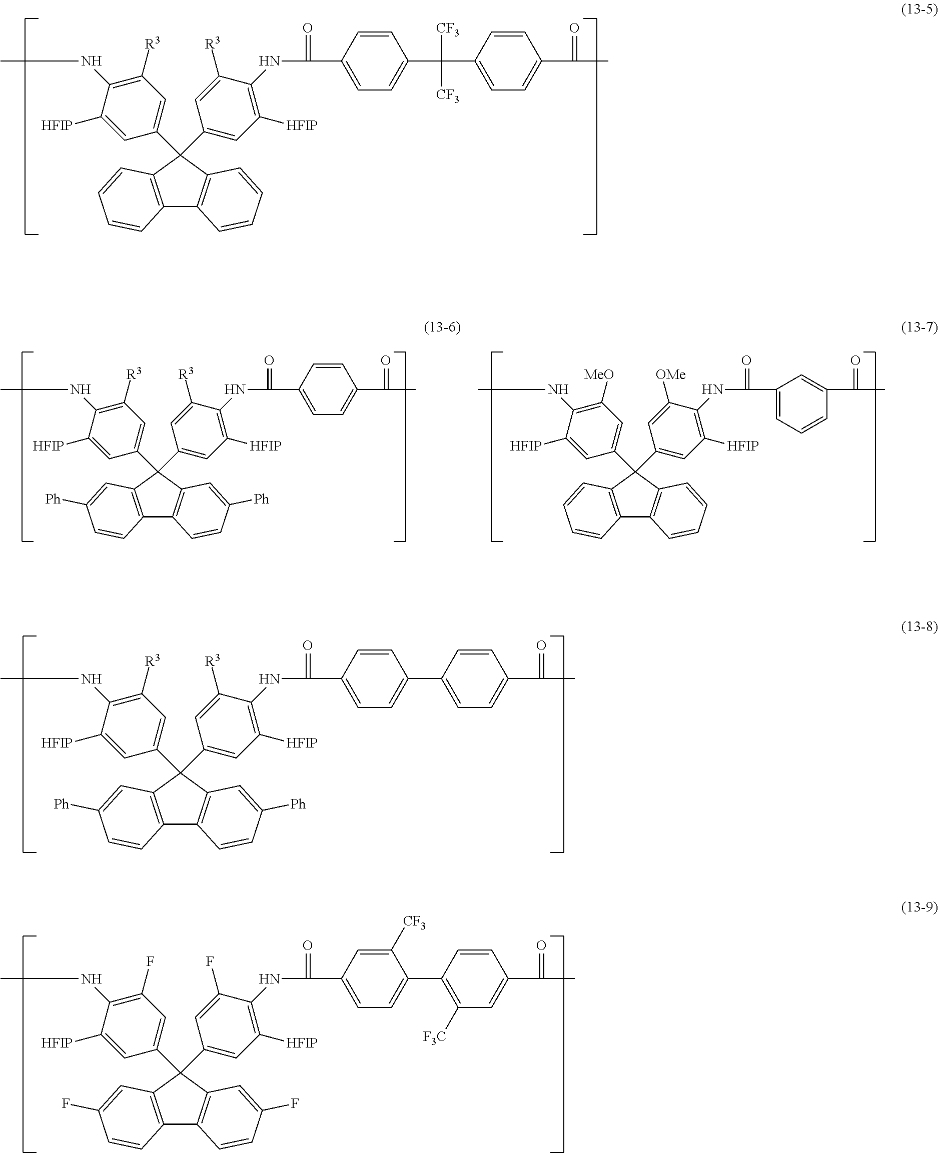

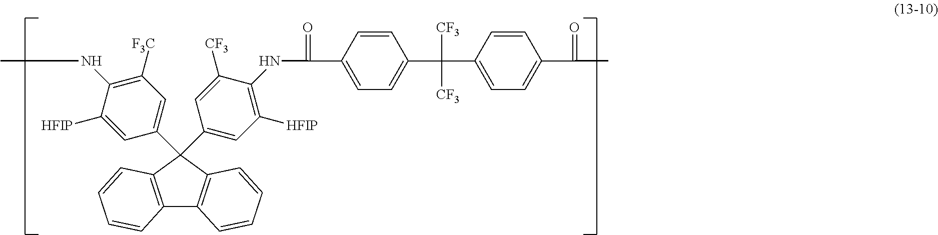

In the formulas (13-1) to (13-10), R³ represents a hydrogen atom or a C₁-C₄ alkyl group; Me represents a methyl group; Ph represents a phenyl group; and HFIP has the same meaning as in the formula (2).

As the polyamide compound, particularly preferred are those having a repeating unit of any of the formulas (13-1) to (13-5) in view of the availability of the fluorenediamine as the raw material for the synthesis of the HFIP-containing fluorenediamine.

The weight-average molecular weight of the polyamide compound (6) is preferably 10,000 or greater, more preferably 20,000 or greater. Further, the weight-average molecular weight of the polyamide compound (6) is preferably 500,000 or smaller, more preferably 300,000 or smaller. If the weight-average molecular weight of the polyamide compound is smaller than 10,000, the resulting polymer film is poor in strength. The resulting polymer solution becomes too high in viscosity so that it is difficult to handle the polymer solution if the weight-average molecular weight of the polyamide compound exceeds 500,000. Herein, the weight-average molecular weight refers to a value measured in term of standard polystyrene by gel permeation chromatography (abbreviated as “GPC”) (the same applies to the following). The conditions for measurement of the molecular weight will be explained later in the following examples.

There is no particular limitation on the synthesis process of the polyamide compound (6). It is feasible to adopt any known process for synthesis of a polyamide compound from a diamine compound and a carboxylic acid or carboxylic acid derivative. For example, a composition containing the HFIP-containing fluorenediamine (2) and the dicarboxylic acid or dicarboxylic acid derivative of the formula (10) or (11) can be reacted by a process in which the reaction substrates are molten together at 150° C. or higher and undergo condensation polymerization in the absence of a solvent, a process in which the reaction substrates undergo condensation polymerization in a solvent at 150° C. or higher, or a process in which the reaction substrates undergo polymerization in a solvent at −20 to 80° C.

As mentioned above, a solvent can be used in the synthesis reaction of the polyamide compound (6). There is no particular limitation on the kind of the solvent used as long as the solvent is capable of dissolving therein the reaction substrates and is not frozen at the reaction temperature. An amide solvent, an aromatic solvent, a halogenated solvent, a lactone solvent etc. is usable as the solvent. Examples of the solvent are N,N-dimethylformamide, N,N-dimethylacetamide, N,N-dimethylformamide, hexamethylphosphoric triamide, N-methyl-2-pyrrolidone, benzene, anisole, diphenyl ether, nitrobenzene, benzonitrile, chloroform, dichloromethane, 1,2-dichloroethane, 1,1,2,2-tetrachloroethane, γ-butyrolactone, γ-valerolactone, δ-valerolactone, γ-caprolactone, ε-caprolactone and α-methyl- γ-butyrolactone. These solvents can be used solely or in the form of a mixture of two or more thereof.

Further, it is effective to perform the reaction in the coexistence of an acid receptor such as pyridine or triethylamine with the solvent.

[Polyamide compound (7)]

As a polymer of the HFIP-containing fluorenediamine according to the present invention, the polyamide compound (7) is synthesized by reaction of the HFIP-containing fluorenediamine (2) with a tetracarboxylic dianhydrate of the formula (14).

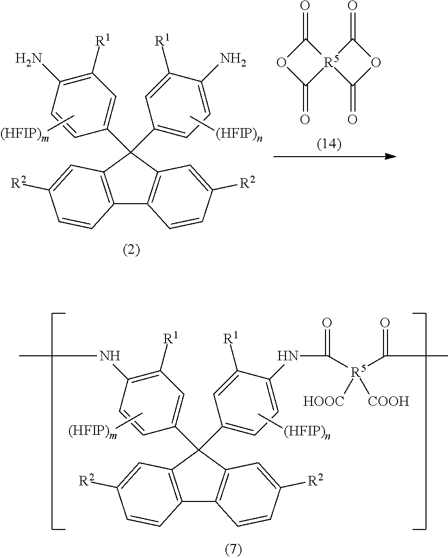

In the above formulas, R¹, R², HFIP, m and n have the same meanings as those in the formula (2); R⁵ represents a tetravalent organic group having one or more kinds selected from the group consisting of alicyclic, aromatic and alkylene groups and may contain a fluorine atom, a chlorine atom, an oxygen atom, a sulfur atom or a nitrogen atom; and any number of hydrogen atoms in R⁵ may be substituted by an alkyl group, a fluoroalkyl group, a carboxyl group, a hydroxy group or a cyano group.

As the tetracarboxylic dianhydrate of the formula (14), there can be used any of those having an aliphatic structure, an alicyclic structure or an aromatic structure in R⁵. Examples of the tetracarboxylic dianhydrate of the formula (14) are benzenetetracarboxylic dianhydrate (pyromellitic dianhydrate), trifluoromethylbenzenetetracarboxylic dianhydrate, bistrifluoromethylbenzenetetracarboxylic dianhydrate, difluorobenzenetetracarboxylic dianhydrate, naphthalenetetracarboxylic dianhydrate, biphenyltetracarboxylic dianhydrate, terphenyltetracarboxylic dianhydrate, 1,1-bis(3,4-dicarboxyphenyl)ketonic dianhydrate, oxydiphthalic dianhydrate, bicycle(2,2,2)octo-7-ene-2,3,5,6-tetracarboxylic dianhydrate, 2,2-bis(3,4-dicarboxyphenyl)hexafluoropropanoic dianhydrate, 2,3,4,5-thiophenetetracarboxylic dianhydrate, 2,5,6,2′,5′,6′-hexafluoro-3,3′,4,4′-biphenyltetracarboxylic dianhydrate, bis(3,4-dicarboxyphenyl)sulfonic dianhydrate and 3,4,8,10-perylenetetracarboxylic dianhydrate. These dianhydrates can be used solely or in combination of two or more kinds thereof.

The polyamide compound (7) is preferably a polyamide compound having at least a repeating unit of either one of the following formulas (15-1) to (15-6).

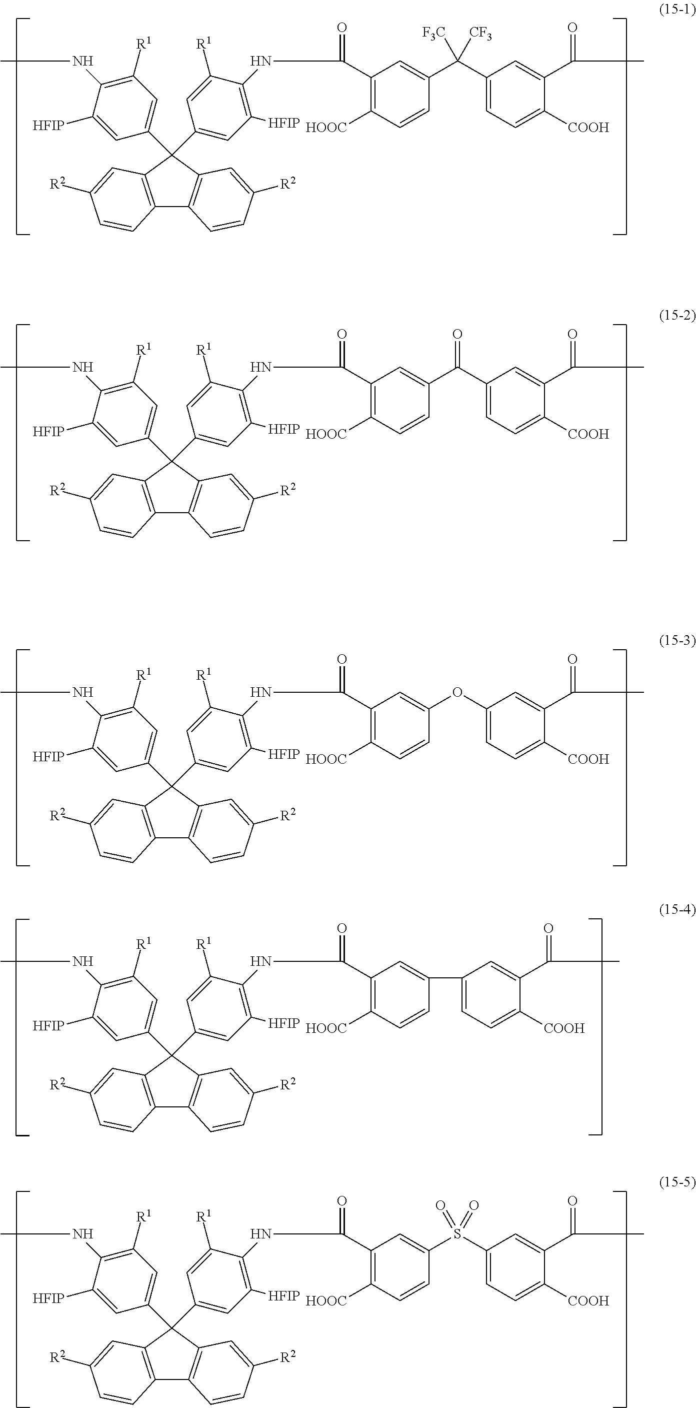

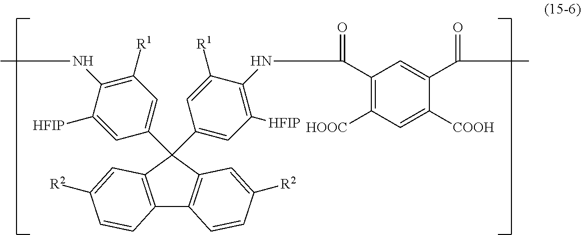

In the formulas (15-1) to (15-6), R¹, R² and HFIP have the same meanings as those in the formula (2).

Specific examples of the polyamide compound are those each having at least a repeating unit of either one of the following formulas (16-1) to (16-12).

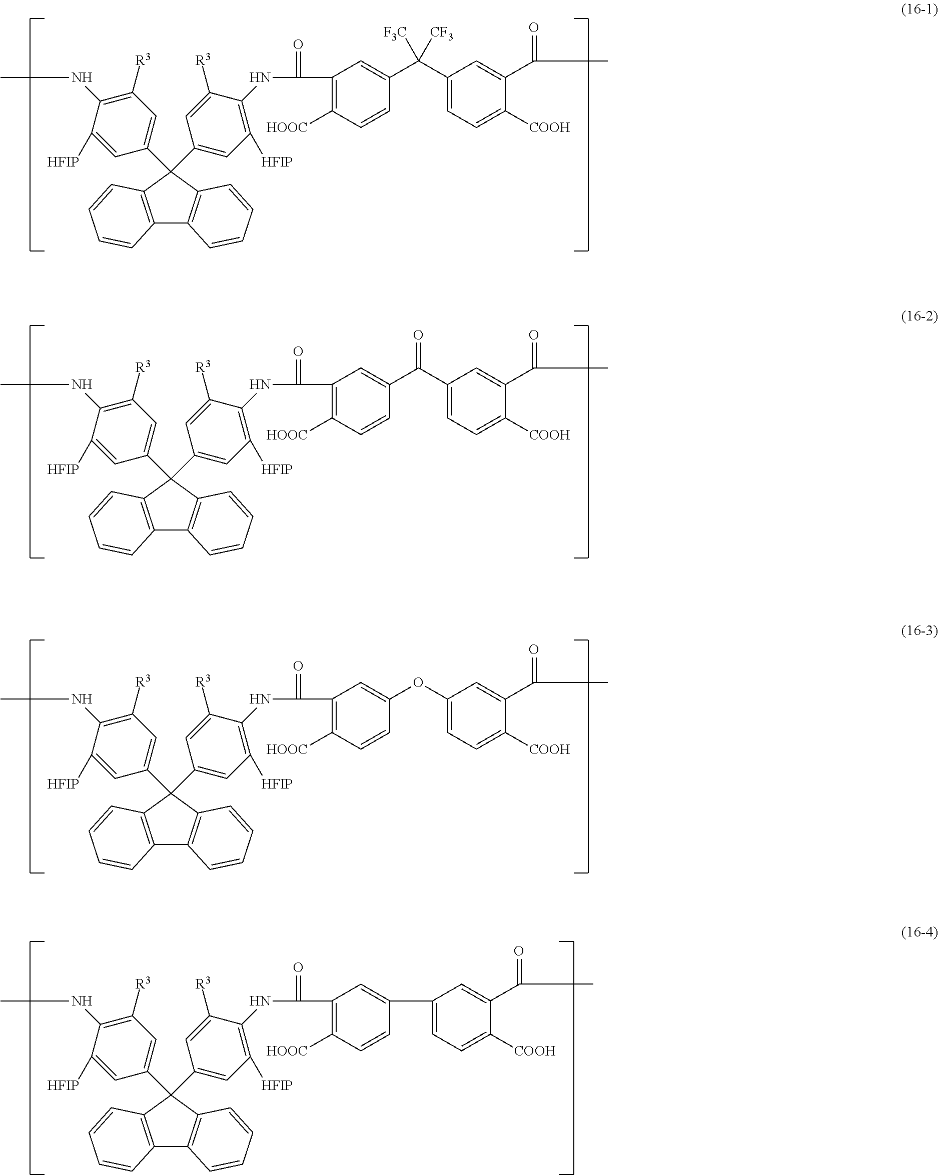

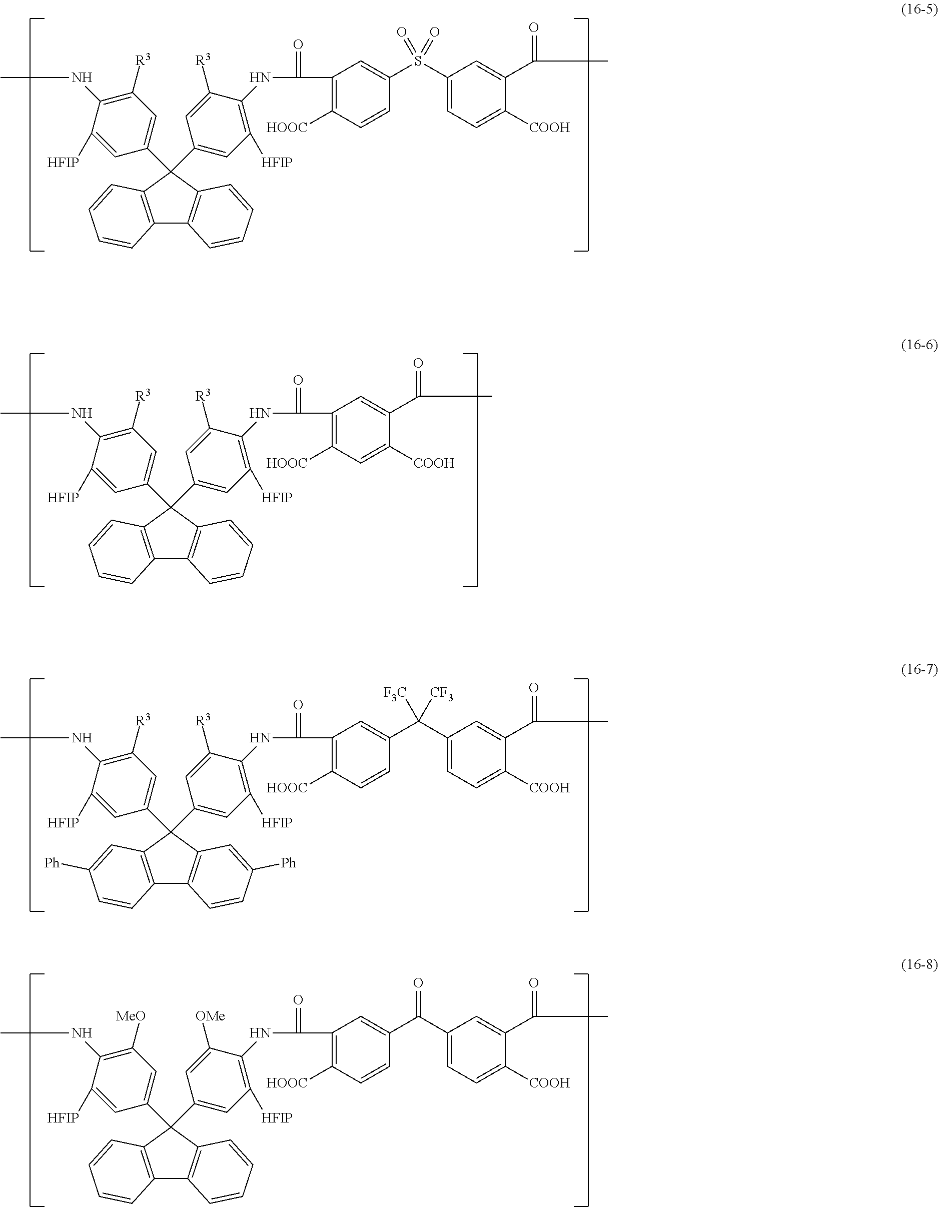

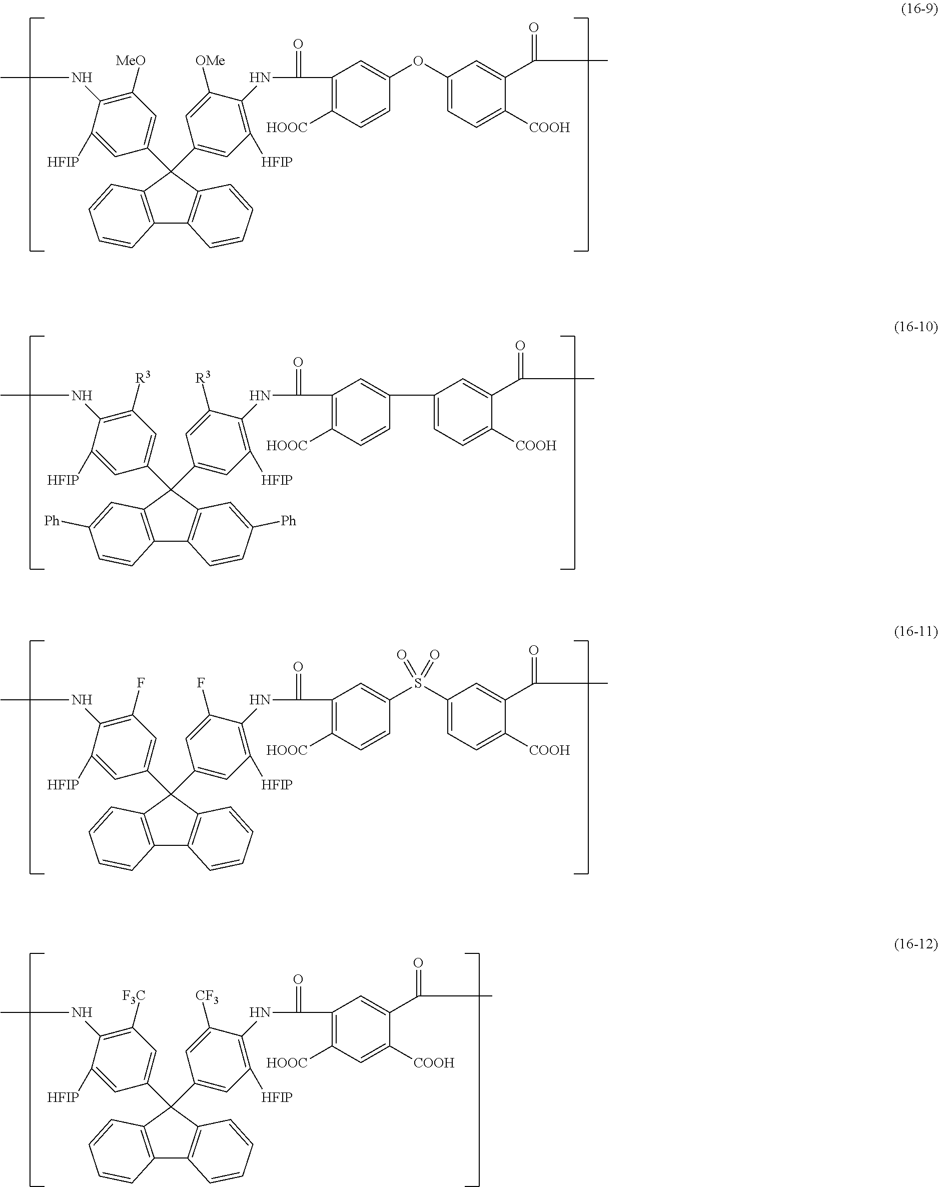

In the formulas (16-1) to (16-12), R³ represents a hydrogen atom or a C₁-C₄ alkyl group; Me represents a methyl group; Ph represents a phenyl group; and HFIP has the same meaning as in the formula (2).

As the polyamide compound, particularly preferred are those having a repeating unit of any of the formulas (16-1) to (16-6) in view of the availability of the fluorenediamine as the raw material for the synthesis of the HFIP-containing fluorenediamine.

The weight-average molecular weight of the polyamide compound (7) is preferably 10,000 or greater, more preferably 20,000 or greater. Further, the weight-average molecular weight of the polyamide compound (7) is preferably 500,000 or smaller, more preferably 300,000 or smaller. If the weight-average molecular weight of the polyamide compound is smaller than 10,000, the resulting polymer film is poor in strength. The resulting polymer solution becomes too high in viscosity so that it is difficult to handle the polymer solution if the weight-average molecular weight of the polyamide compound exceeds 500,000.

There is no particular limitation on the synthesis process of the polyamide compound (7). For example, a composition containing the HFIP-containing fluorenediamine (2) and the dicarboxylic acid derivative of the formula (14) can be reacted by a process in which the reaction substrates are molten together at 150° C. or higher and undergo condensation polymerization in the absence of a solvent, a process in which the reaction substrates undergo condensation polymerization in a solvent at 150° C. or higher, or a process in which the reaction substrates undergo polymerization in a solvent at −20 to 80° C.

As mentioned above, a solvent can be used in the synthesis reaction of the polyamide compound (7). There is no particular limitation on the kind of the solvent used as long as the solvent is capable of dissolving therein the reaction substrates and is not frozen at the reaction temperature. An amide solvent, an aromatic solvent, a halogenated solvent, a lactone solvent etc. is usable as the solvent. Examples of the solvent are N,N-dimethylformamide, N,N-dimethylacetamide, N,N-dimethylformamide, hexamethylphosphoric triamide, N-methyl-2-pyrrolidone, benzene, anisole, diphenyl ether, nitrobenzene, benzonitrile, chloroform, dichloromethane, 1,2-dichloroethane, 1,1,2,2-tetrachloroethane, ε-butyrolactone, γ-valerolactone, δ-valerolactone, γ-caprolactone, ε-caprolactone and α-methyl-γ-butyrolactone. These solvents can be used solely or in the form of a mixture of two or more thereof.

[Polyimide Compound]

As a polymer of the HFIP-containing fluorenediamine according to the present invention, the polyimide compound (8) is synthesized from the polyamide compound (7).

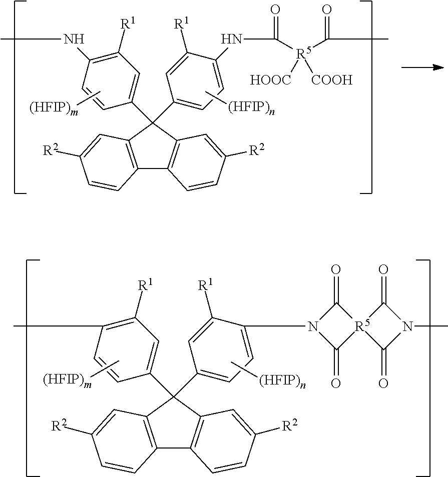

In the above formulas, R¹, R², HFIP, m and n have the same meanings as those in the formula (2); R⁵ represents a tetravalent organic group having one or more kinds selected from the group consisting of alicyclic, aromatic and alkylene groups and may contain a fluorine atom, a chlorine atom, an oxygen atom, a sulfur atom or a nitrogen atom; and any number of hydrogen atoms in R³ may be substituted by an alkyl group, a fluoroalkyl group, a carboxyl group, a hydroxy group or a cyano group.

The polyimide compound (8) is preferably a polyimide compound having at least a repeating unit of either one of the following formulas (17-1) to (17-6).

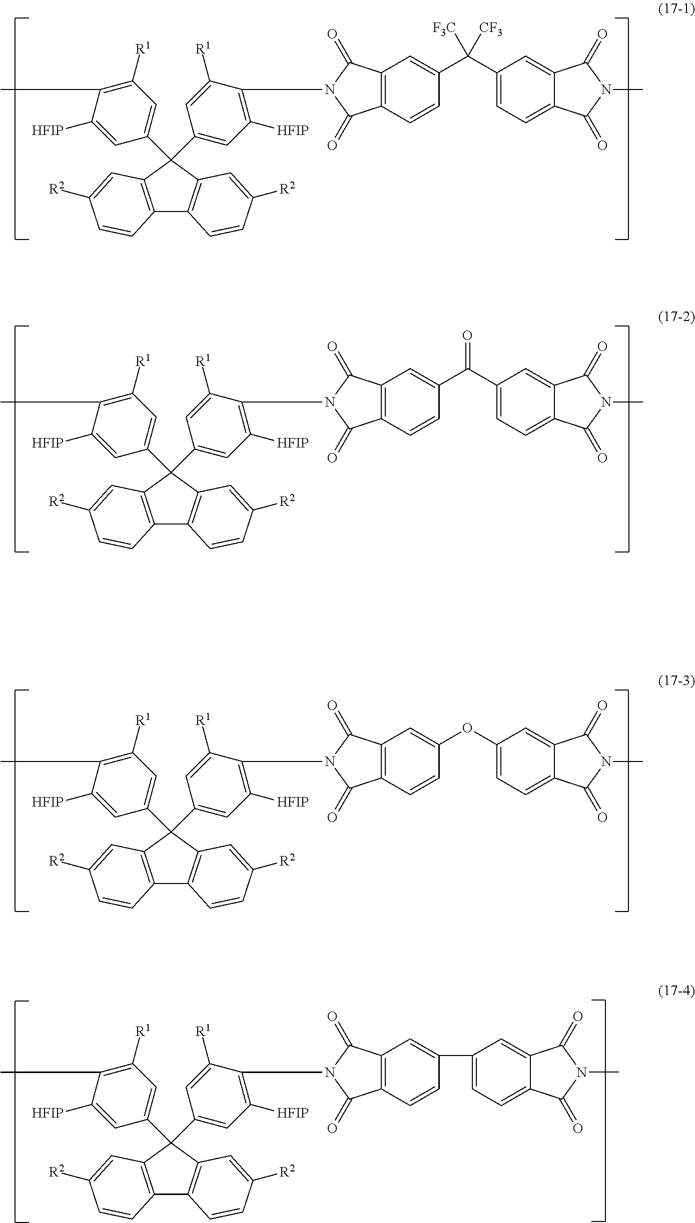

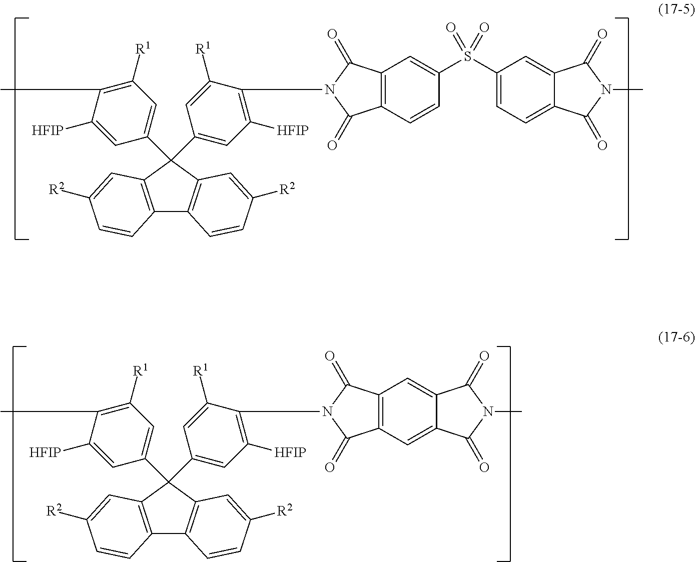

In the formulas (17-1) to (17-6), R¹, R² and HFIP have the same meanings as those in the formula (2).
Specific examples of the polyimide compound are those each having at least a repeating unit of either one of the following formulas (18-1) to (18-12).

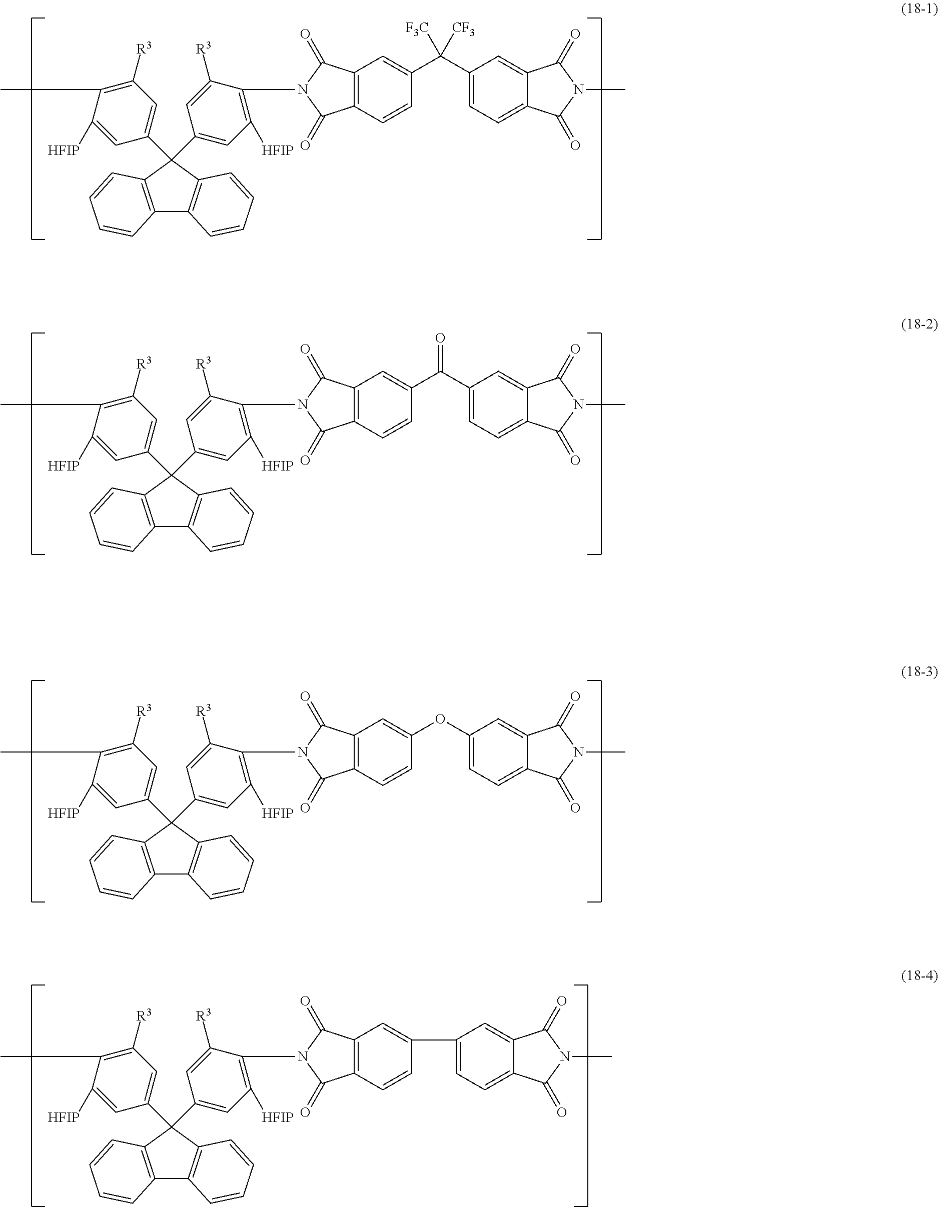

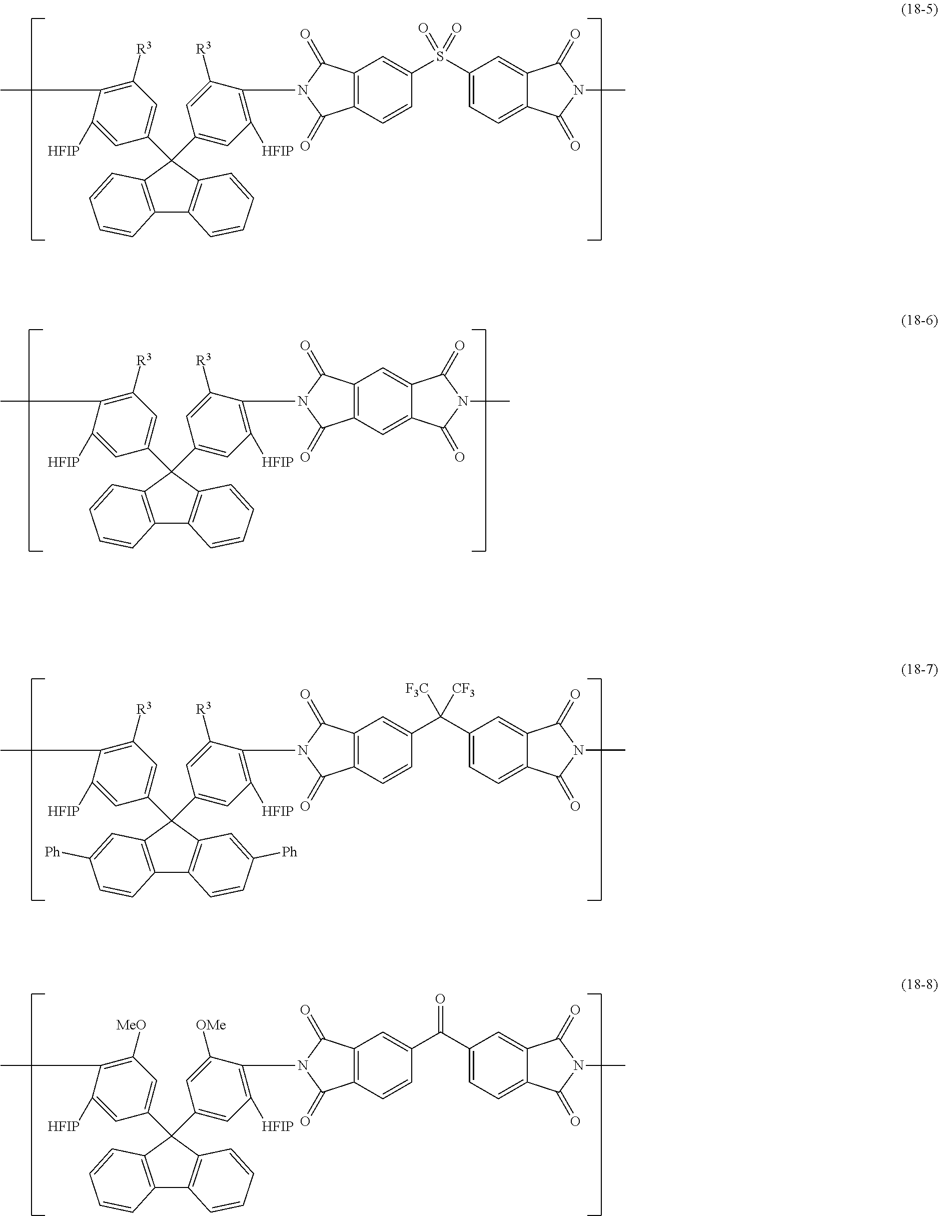

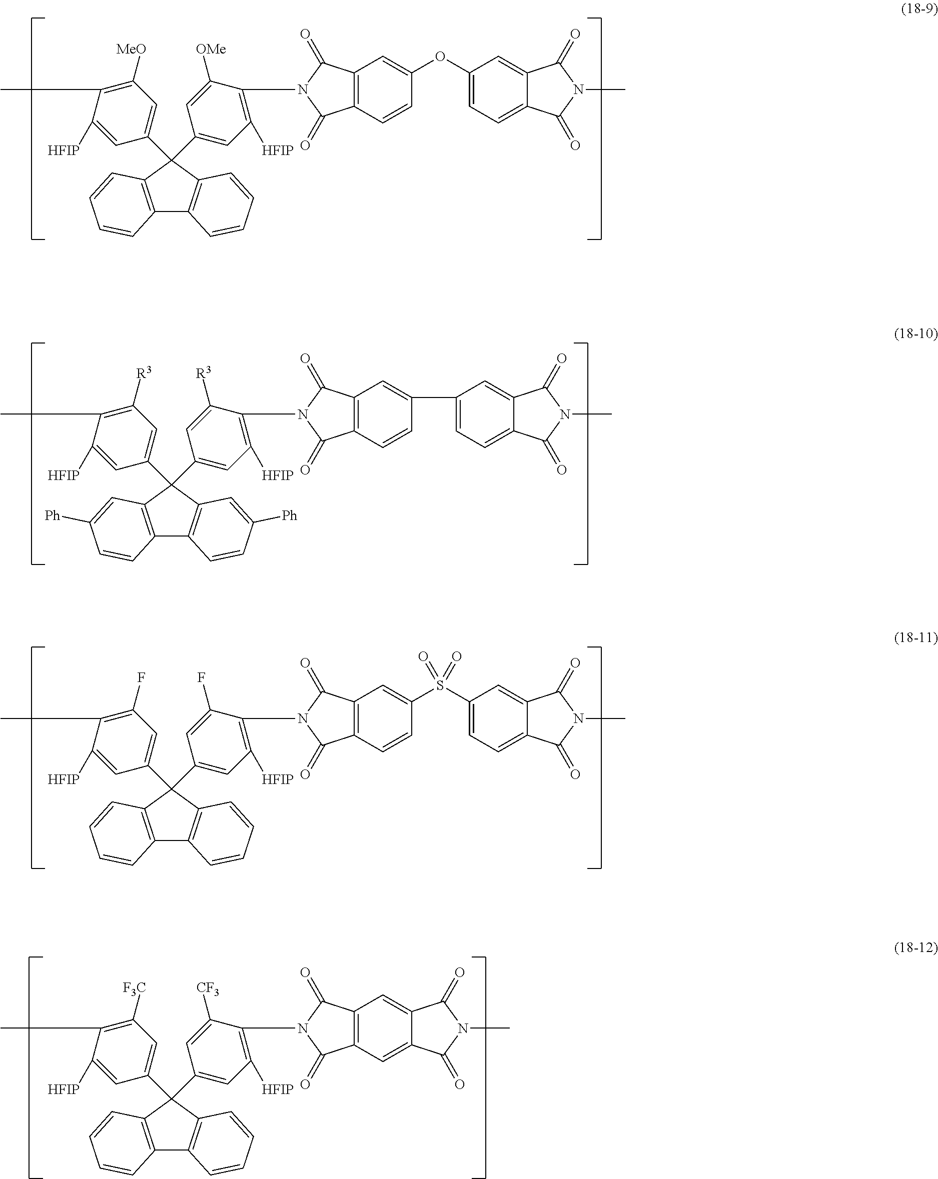

In the formulas (18-1) to (18-12), R³ represents a hydrogen atom or a C₁-C₄ alkyl group; Me represents a methyl group; Ph represents a phenyl group; and HFIP has the same meaning as in the formula (2).

As the polyimide compound, particularly preferred are those having a repeating unit of any of the formulas (18-1) to (18-6) in view of the availability of the fluorenediamine as the raw material for the synthesis of the HFIP-containing fluorenediamine.

The weight-average molecular weight of the polyimide compound (8) is preferably 10,000 or greater, more preferably 20,000 or greater. Further, the weight-average molecular weight of the polyamide compound (7) is preferably 500,000 or smaller, more preferably 300,000 or smaller. If the weight-average molecular weight of the polyamide compound is smaller than 10,000, the resulting polymer film is poor in strength. The resulting polymer solution becomes too high in viscosity so that it is difficult to handle the polymer solution if the weight-average molecular weight of the polyamide compound exceeds 500,000.

The synthesis reaction of the polyimide compound (8), that is, the dehydration ring-closing reaction of the polyamide compound (7) is performed under accelerated reaction conditions such as by heating or by combination of heating and adding an additive such as acid or base.

In general, a solution of the HFIP-containing polyimide compound can be obtained by imidization of a solution of the polyamide compound (7) under high-temperature conditions of 150 to 250° C. At this time, pyridine, triethylamine, acetic anhydride or the like may be added as an additive. The concentration of the HFIP-containing polyimide compound in the solution is preferably 5 to 50 mass %. If the polyimide concentration is lower than 5 mass %, the solution is too weak for industrial practical use. If the polyimide concentration is higher than 50 mass %, it is difficult to dissolve the polyimide compound. The concentration of the HFIP-containing polyimide compound in the solution is more preferably 10 to 40 mass %.

There is no particular limitation on the kind of the solvent used for the preparation of the solution of the polyamide compound (7) as long as the solvent is capable of dissolving therein the reaction substrate. Examples of the solvent are: amide solvents such as N,N-dimethylformamide, N,N-dimethylacetamide, hexamethylphosphoric triamide and N-methyl-2-pyrrolidone; aromatic solvents such as benzene, anisole, diphenyl ether, nitrobenzene and benzonitrile; halogenated solvents such as chloroform, dichloromethane, 1,2-dichloroethane and 1,1,2,2-tetrachloroethane; and lactone solvents such as γ-butyrolactone, γ-valerolactone, δ-valerolactone, γ-caprolactone, ε-caprolactone and α-methyl-γ-butyrolactone. These solvents can be used solely or in the form of a mixture of two or more thereof.

Alternatively, the reaction solution immediately after the synthesis reaction of the polyamide compound (7) may be subjected as it is to dehydration ring-closing reaction as a process for synthesis of the polyimide compound (8). The above-mentioned temperature range, additive, concentration and solvent are also applicable to this process.

[Fluorine-Containing Heterocyclic Polymer Compound]

As a polymer of the HFIP-containing fluorenediamine according to the present invention, the fluorine-containing heterocyclic polymer compound (9) is synthesized by dehydration cyclization of a polyamide compound having at least a repeating unit of the formula (19) (sometimes referred to as “polyamide compound (19)”).

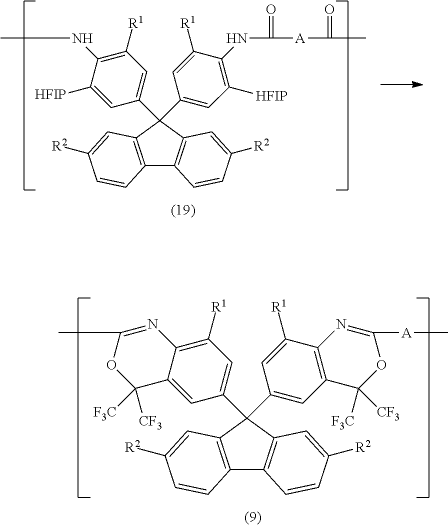

In the above formulas, R¹, R², HFIP, m and n have the same meanings as those in the formula (2); A represents a divalent organic group having one or more kinds selected from the group consisting of alicyclic, aromatic and alkylene groups and may contain an oxygen atom, a sulfur atom or a nitrogen atom; and any number of hydrogen atoms in A may be substituted by an alkyl group, a fluorine atom, a chlorine atom, a fluoroalkyl group, a carboxyl group, a hydroxy group or a cyano group.
The dehydration cyclization reaction proceeds to form a heterocyclic ring as indicated by the following reaction scheme.

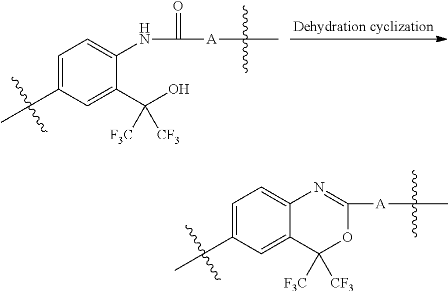

In the above scheme, A represents a divalent organic group having one or more kinds selected from the group consisting of alicyclic, aromatic and alkylene groups and may contain an oxygen atom, a sulfur atom or a nitrogen atom; any number of hydrogen atoms in A may be substituted by an alkyl group, a fluorine atom, a chlorine atom, a fluoroalkyl group, a carboxyl group, a hydroxy group or a cyano group; and the line intersecting a wavy line represents a bonding position.

The fluorine-containing heterocyclic polymer compound (9) is preferably a fluorine-containing heterocyclic polymer compound having at least a repeating unit of either one of the following formulas (20-1) to (20-5).

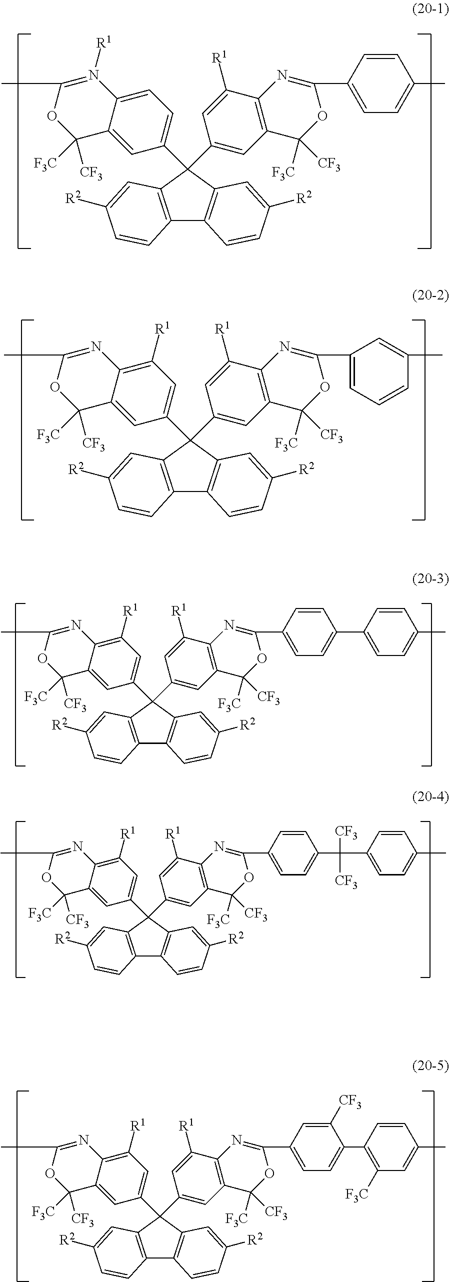

In the formulas (20-1) to (20-5), R¹, R² and HFIP have the same meanings as those in the formula (2).
Specific examples of the fluorine-containing heterocyclic polymer compound are those each having at least a repeating unit of either one of the following formulas (21-1) to (21-10).

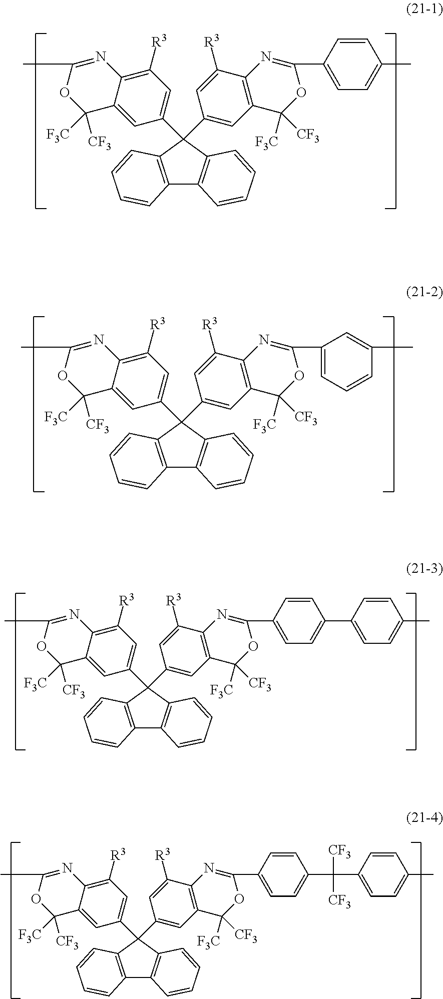

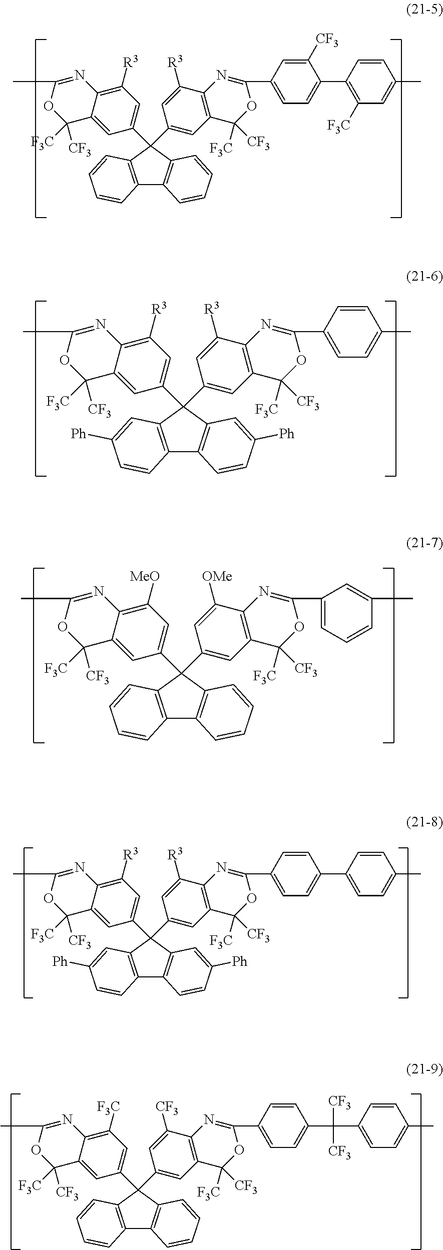

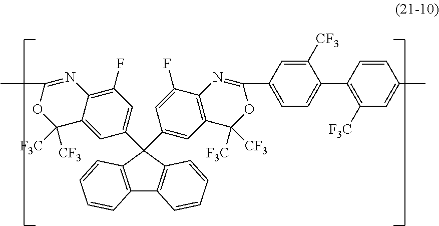

In the formulas (21-1) to (21-10), R³ represents a hydrogen atom or a C₁-C₁ alkyl group; Me represents a methyl group; and Ph represents a phenyl group.

As the fluorine-containing heterocyclic polymer compound, particularly preferred are those having a repeating unit of any of the formulas (21-1) to (21-5) in view of the availability of the fluorenediamine as the raw material for the synthesis of the HFIP-containing fluorenediamine.

The weight-average molecular weight of the fluorine-containing heterocyclic polymer compound (9) is preferably 10,000 or greater, more preferably 20,000 or greater. Further, the weight-average molecular weight of the fluorine-containing heterocyclic polymer compound (9) is preferably 500,000 or smaller, more preferably 300,000 or smaller. If the weight-average molecular weight of the fluorine-containing heterocyclic polymer compound is smaller than 10,000, the resulting polymer film is poor in strength. The resulting polymer solution becomes too high in viscosity so that it is difficult to handle the polymer solution if the weight-average molecular weight of the fluorine-containing heterocyclic polymer compound exceeds 500,000.

The reaction conditions such as heating, the use of an acid catalyst or a base catalyst and the like can be selected as appropriate for the synthesis of the fluorine-containing heterocyclic polymer compound by the dehydration cyclization reaction of the polyamide compound (19).

For example, it is feasible to adopt the conditions of similar dehydration cyclization reaction disclosed in Patent Documents 6 and 7.

By way of example, the fluorine-containing heterocyclic polymer compound (9) can be obtained by heating the polyamide compound (19) in a solvent at 150 to 250° C. in such a manner that the polyamide compound (19) undergoes dehydration cyclization. At this time, pyridine, triethylamine, acetic anhydride or the like may be added in order to perform the reaction efficiently.

As mentioned above, a solvent can be used in the synthesis reaction of the fluorine-containing heterocyclic polymer compound (9). There is no particular limitation on the kind of the solvent used as long as the solvent is capable of dissolving therein the reaction substrate and is not frozen at the reaction temperature. An amide solvent, an aromatic solvent, a halogenated solvent, a lactone solvent etc. is usable as the solvent Examples of the solvent are N,N-dimethylformamide, N,N-dimethylacetamide, N,N-dimethylformamide, hexamethyiphosphoric triamide, N-methyl-2-pyrrolidone, benzene, anisole, diphenyl ether, nitrobenzene, benzonitrile, chloroform, dichloromethane, 1,2-dichloroethane, 1,1,2,2-tetrachloroethane, γ-butyrolactone, γ-valerolactone, δ-valerolactone, γ-caprolactone, ε-caprolactone and α-methyl-γ-butyrolactone. These solvents can be used solely or in the form of a mixture of two or more thereof. It is effective to perform the reaction in the coexistence of an acid receptor such as pyridine or triethylamine with the solvent.

[Polymerization Component]

In order to improve the heat resistance of the polymer compound and secure the solubility of the polymer compound in any desired organic solvent, it is feasible to add an HFIP-free diamine compound as a polymerization component in the synthesis of the polyamide compound (6), the polyamide compound (7), the polyimide compound (8) and the fluorine-containing heterocyclic polymer compound (9) from the HFIP-containing fluorenediamine (2).

Example of such a diamine compound are 3,5-diaminobenzotrifluoride, 2,5-diaminobenzotrifluoride, 3,3′-bistrifluoromethyl-4,4′-diaminobiphenyl, 3,3′-bistrifluoromethyl-5,5′-diaminobiphenyl, bis(trifluoromethyl)-4,4′-diaminophenyl, bis(fluoroalkyl)-4,4′-diaminodiphenyl, dichloro-4,4′-diaminodiphenyl, dibromo-4,4′-diaminodiphenyl, bis(fluoroalkoxy)-4,4′-diaminodiphenyl, diphenyl-4,4′-diaminodiphenyl, 4,4′-bis(4-aminotetrafluorophenoxy)tetrafluorobenzene, 4,4′-bis(4-aminotetrafluorophenoxy)octafluorobiphenyl, 4,4′-binaphthylamine, o-, m- or p-phenylenediamine, 2,4-diaminotoluene, 2,5-diaminotoluene, 2,4-diaminoxylene, 2,4-diaminodurene, 1,4-xylylenediamine, dimethyl-4,4′-diaminodiphenyl, dialkyl-4,4′-diaminodiphenyl, dimethoxy-4,4′-diaminodiphenyl, diethoxy-4,4′-diaminodiphenyl, 4,4′-diaminodiphenylmethane, 3,3′-dimethyl-diaminodiphenylmethane, 3,3′-diethyl-diaminodiphenylmethane, 9,9-bis(4-aminophenyl)fluorene, 4,4′-diaminodiphenyl ether, 3,4′-diaminodiphenyl ether, 4,4′-diaminodiphenyl sulfide, 3,4′-diaminodiphenyl sulfide, 4,4′-diaminodiphenylsulfone, 3,3′-diaminodiphenylsulfone, 4,4′-diaminobenzophenone, 3,3′-diaminobenzophenone, 1,3-bis(3-aminophenoxy)benzene, 1,3-bis(4-aminophenoxy)benzene, 1,4-bis(4-aminophenoxy)benzene, 4,4′-bis(4-aminophenoxy)biphenyl, bis(4-(3-aminophenoxy)phenyl)sulfone, bis(4-(4-aminophenoxy)phenyl)sulfone, 2,2-bis(4-(4-aminophenoxy)phenyl)propane, 2,2-bis(4-(4-aminophenoxy)phenyl)hexafluoropropane, 2,2-bis(4-(3-aminophenoxy)phenyl)propane, 2,2-bis(4-(3-aminophenoxy)phenyl)hexafluoropropane, 2,2-bis(4-(4-amino-2-trifluoromethylphenoxy)phenyehexafluoropropane, 2,2-bis(4-(3-amino-5-trifluoromethylphenoxy)phenyl)hexafluoropropane, 2,2-bis(4-aminophenyl)hexafluoropropane, 2,2-bis(3-aminophenyl)hexafluoropropane, 2,2-bis(3-amino-4-hydroxyphenyl)hexafluoropropane, 2,2-bis(3-amino-4-methylphenyl)hexafluoropropane, 4,4′-bis(4-aminophenoxy)octafluorobiphenyl and 4,4′-diaminobenzanilide. These diamine compounds can be used solely or in combination of two or more kinds thereof

[Use of Polymer Compound]

The polymer compound according to the present invention can be used in the form of a varnish in which the polymer is dissolved in an organic solvent, a power, a film or a solid. Any additive such as oxidation stabilizer, filler, silane coupling agent, photosensitizer, photopolymerization initiator, sensitizer etc. can be added as needed to the polymer compound without any problem.

In the case of using the polymer compound in varnish form, it is feasible to apply the varnish of the polymer compound to a substrate e.g. glass substrate, silicon wafer, metal substrate, metal oxide substrate, ceramic substrate or resin substrate by any ordinary process such as spin coating, spray coating, flow coating, impregnation coating or brush painting.

### EXAMPLES

Hereinafter, the present invention will be described in more detail below by way of the following examples. The following examples are illustrative and are not intended to limit the present invention thereto.

The properties of HFIP-containing fluorenediamines according to the present invention and polymer compounds synthesized therefrom were evaluated by the following procedures.

[NMR (Nuclear Magnetic Resonance) Analysis]

The structure of the respective compounds was identified by ¹H-NMR analysis and ¹⁹F-NMR analysis using a NMR (nuclear magnetic resonance) analyzer (JNM-AL400 or JNM-ECA400 manufactured by JEOL Ltd.) with a resonance frequency of 400 MHz.

[Weight-Average Molecular Weight Determination]

The weight-average molecular weight (sometimes referred to as “molecular weight Mw”) of the respective compounds was determined by gel permeation chromatograph (HLC-8320 manufactured by Tosoh Corporation, solvent: tetrahydrofuran) using polystyrene as a standard.

### Example 1

### Synthesis of HFIP-Containing Fluorenediamine of Formula (22) (FHIP-FL)

Into a 300-mL autoclave, 9,9-bis(4-aminophenyl)fluorene (25 g, 0.072 mol; available from Tokyo Chemical Industry Co., Ltd.), hexafluoroacetone trihydrate (70 g, 0.32 mol) and paratoluenesulfonic acid monohydrate (0.68 g, 3.6 mmol) were placed. The autoclave was sealed. The resulting reaction solution inside the autoclave was stirred for 17 hours while the autoclave was heated at 130° C. in an oil bath. Then, the autoclave was cooled down to room temperature. (The term “room temperature” refers to a temperature of atmosphere without heating or cooling and generally ranges from about 15 to 30° C. The same applies to the following.) To the reaction solution, toluene (50 g) was added. The thus-formed solid was filtered out, washed with water and further washed with chloroform (100 ml). The washed solid was filtered out and dried under vacuum. There was thus obtained a white powder (11.6 g, 0.017 mol, yield: 25%). It was confirmed by NMR analysis that the white powder was HFIP-containing fluorenediamine of the formula (22) (sometimes referred to as “HFIP-FL”).

### [NMR Analysis]

¹H-NMR (DMSO-d₆): δ 9.19 (brs, 2H), 7.87 (d, J=7.6 Hz, 2H), 7.35 (m, 2H), 7.26 (m, 4H), 7.02 (s, 2H), 6.80 (dd, J=8.5, 1.7 Hz, 2H), 6.60 (d, J=8.5 Hz, 2H), 5.55 (brs, 4H).

¹⁹F-NMR (DMSO-d₆): δ −72.65 (s).

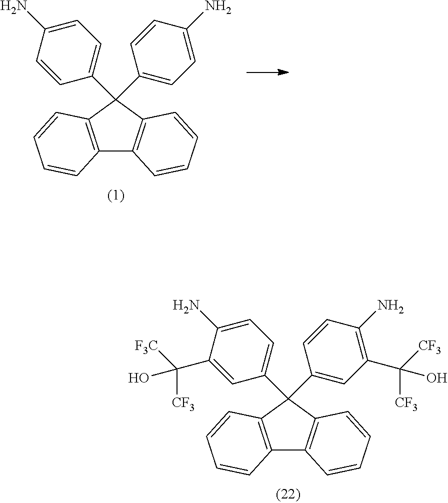 [SMILES: Nc1ccc(C2(c3ccc(N)cc3)c3ccccc3-c3ccccc32)cc1 ; Nc1ccc(C2(c3ccc(N)c(C(O)(C(F)(F)F)C(F)(F)F)c3)c3ccccc3-c3ccccc32)cc1C(O)(C(F)(F)F)C(F)(F)F]

### Example 2

### Synthesis of HFIP-Containing Fluorenediamine of Formula (23) (HFIP-MeFL)

Into a 300-mL autoclave, 9,9-bis(4-amino-3-methylphenyl)fluorene (70 g, 0.186 mol), hexafluoroacetone trihydrate (220 g, 1.000 mol) and paratoluenesulfonic acid monohydrate (1.77 g, 9.3 mmol) were placed. The autoclave was sealed. The resulting reaction solution inside the autoclave was stirred for 24 hours while the autoclave was heated at 130° C. in an oil bath. Then, the autoclave was cooled down to room temperature. To the reaction solution, methanol (200 mL) was added. The thus-formed solid was filtered out and washed with chloroform (100 ml). The washed solid was filtered out and dried under vacuum. There was thus obtained a white powder (15.2 g, 0.022 mol, yield: 20%). It was confirmed by NMR analysis that the white powder was HFIP-containing fluorenediamine of the formula (23) (sometimes referred to as “HFIP-MeFL”).

### [NMR Analysis]

¹H-NMR (DMSO-d₆): δ 10.11 (brs, 2H), 7.86 (d, J=7.2 Hz, 2H), 7.35 (m, 2H), 7.26 (m, 4H), 6.97 (s, 2H), 6.79 (s, 2H), 5.24 (brs, 4H), 1.98 (s, 6H).

¹⁹F-NMR (DMSO-d₆): δ −72.92 (s).

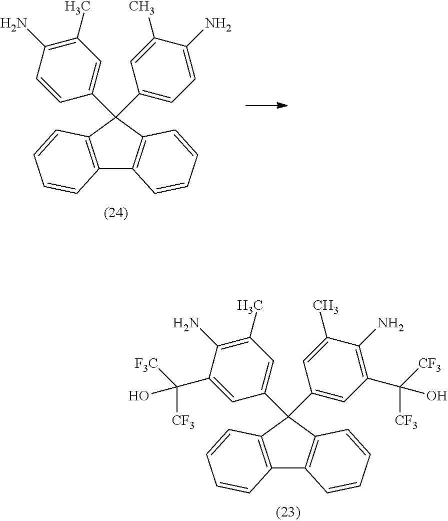 [SMILES: Cc1cc(C2(c3ccc(N)c(C)c3)c3ccccc3-c3ccccc32)ccc1N ; Cc1cc(C2(c3cc(C)c(N)c(C(O)(C(F)(F)F)C(F)(F)F)c3)c3ccccc3-c3ccccc32)cc(C(O)(C(F)(F)F)C(F)(F)F)c1N]

### Example 3

### Synthesis of Polymer P1 by Polymerization of HFIP-FL and 6FDA

Into a 300-mL three-neck flask, HFIP-FL (10.01 g, 14.7 mmol), 4,4′-(hexafluoroisopropylidene)diphthalic anhydride (sometimes referred to as “6FDA”; 6.533 g, 14.7 mmol) and N,N-dimethylacetamide (38.57 g) were placed. The resulting reaction solution was stirred at room temperature for 18 hours under an atmosphere of nitrogen, and then, dropped into a mixed solvent of water and methanol (mixing volume ratio 1:1). The thus-formed white precipitate was recovered by filtration and dried under vacuum at room temperature for 12 hours. There was thus obtained a polymer P1 (yield: 14.89 g, 90%). It was confirmed by GPC measurement that the molecular weight Mw of the polymer P1 was 51,000. In the following scheme, n represents the number of repeating units (the same applies to the following examples).

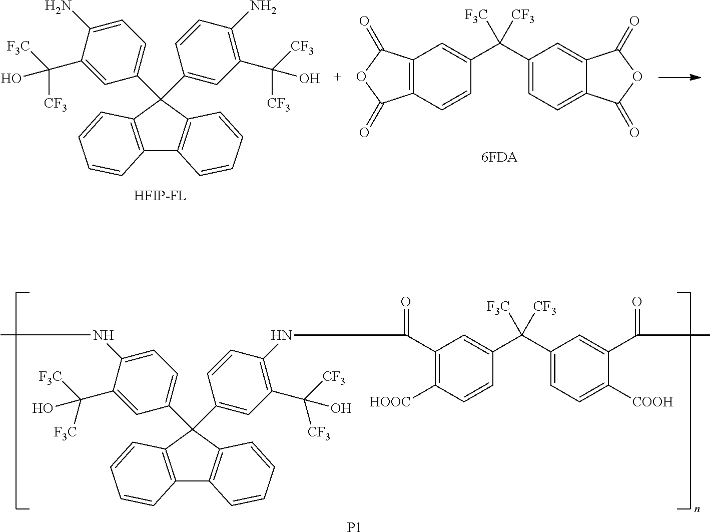

### Example 4

### Synthesis of Polymer P1-1 by Imidization of Polymer P1 and Solvent Solubility Test of Polymer P1-1

In a 100-mL eggplant-shaped flask, the polymer P1 (5 g) was placed together with acetic anhydride (0.998 g, 9.78 mmol) and triethylamine (1.08 g, 10.66 mmol) and dissolved in N,N-dimethylacetamide (20 g). The resulting reaction solution was reacted for 24 hours at 110° C., and then, dropped into a mixed solvent of water and methanol (mixing volume ratio 1:1). The thus-formed white precipitate was recovered by filtration and dried under vacuum at room temperature for 12 hours. There was thus obtained a polymer P1-1 (yield: 4.11 g, 85%). It was confirmed by GPC measurement that the molecular weight Mw of the polymer P1-1 was 31,000. Further, the structure of the polymer P1-1 was identified by observation of absorption peaks at 1780 cm⁻¹ and 1720 cm⁻¹ characteristic to imide group in IR spectrum measurement. The polymer P1-1 was soluble in tetrahydrofuran and in N,N-dimethylformamide. (The solubility of the polymer solid in the solvent was visually checked upon stirring 5 wt % of the polymer solid in the solvent at room temperature. The same applies to the following.)

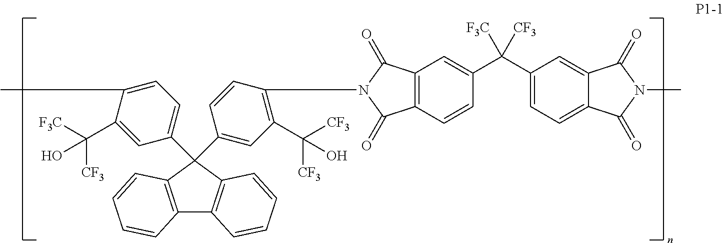

### Example 5

### Film Formation of Polymer P1-1

The polymer P1-1 (3 g) obtained in Example 4 was dissolved in N,N-dimethylacetamide (12 g). The resulting solution was spin-coated onto a glass substrate, and then, heated for 1 hour at 100° C. and for 1 hour at 180° C. assuming that the upper limit of the heating temperature was 180° C. close to the boiling point of N,N-dimethylacetamide. Thus, a coating film of the polymer P1-1 was formed with a uniform thickness of 25 μm on the glass substrate. No separation and crack was visually observed in the coating film.

### Example 6

### Synthesis of Polymer P2 by Polymerization of HFIP-MeFL and Isophthaloyl Chloride

Into a 300-mL three-neck flask, HFIP-MeFL (9.00 g, 12.7 mmol), isophthaloyl chloride (2.52 g, 12.7 mmol) and N,N-dimethylacetamide (46 g) were placed. The resulting reaction solution was stirred at room temperature for 18 hours under an atmosphere of nitrogen, and then, dropped into a mixed solvent of water and methanol (mixing volume ratio 1:1). The thus-formed white precipitate was recovered by filtration and dried under vacuum at room temperature for 12 hours. There was thus obtained a polymer P2 (yield: 8.73 g, 83%). It was confirmed by GPC measurement that the molecular weight Mw of the polymer P2 was 25,000.

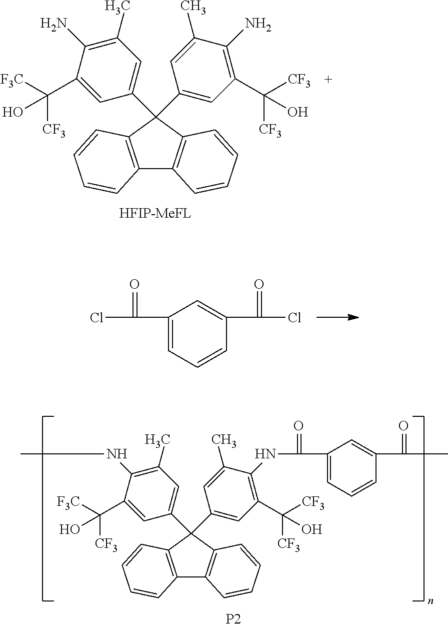

### Example 7

### Synthesis of Polymer P2-1 by Heating Dehydration of Polymer P2 and Solvent Solubility Test of P2-1

The polymer P2 (6 g) was dissolved in N-methylpyrrolidone (20 g). The resulting solution was stirred for 12 hours at 200° C., and then, dropped into a mixed solvent of water and methanol (mixing volume ratio 1:1). The thus-formed white precipitate was recovered by filtration and dried under vacuum at room temperature for 12 hours. There was thus obtained a polymer P2-1 (yield: 3.83 g, 80%). It was confirmed by GPC measurement that the molecular weight Mw of the polymer P2-1 was 23,000. Further, the structure of the polymer P2-1 was identified by observation of an absorption peak at 1650 cm⁻¹ characteristic to C(═N) group in IR spectrum measurement. The polymer P2-1 was soluble in N-methylpyrrolidone.

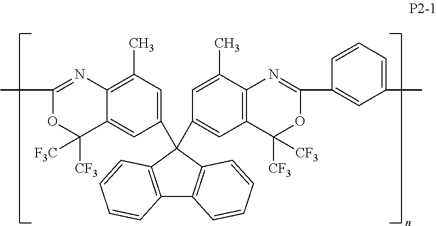

### Example 8

### Film Formation of Polymer P2-1

The polymer P2-1 (3 g) obtained in Example 7 was dissolved in N-methylpyrrolidone (12 g). The resulting solution was spin-coated onto a glass substrate, and then, heated for 1 hour at 100° C. and for 1 hour at 200° C. assuming that the upper limit of the heating temperature was 200° C. close to the boiling point of N-methylpyrrolidone. Thus, a coating film of the polymer P2-1 was formed with a uniform thickness of 23 μm on the glass substrate. No separation and crack was visually observed in the coating film.

### Examples 9 to 13

### Synthesis of Polymers P3 to P7

The following polymers P3 to P7 were each synthesized by polymerization of HFIP-FL or HFIP-MeFL with various tetracarboxylic dianhydride such as PMDA, BPDA, BTDA, DSDA or ODPA in the same manner as in Example 3.

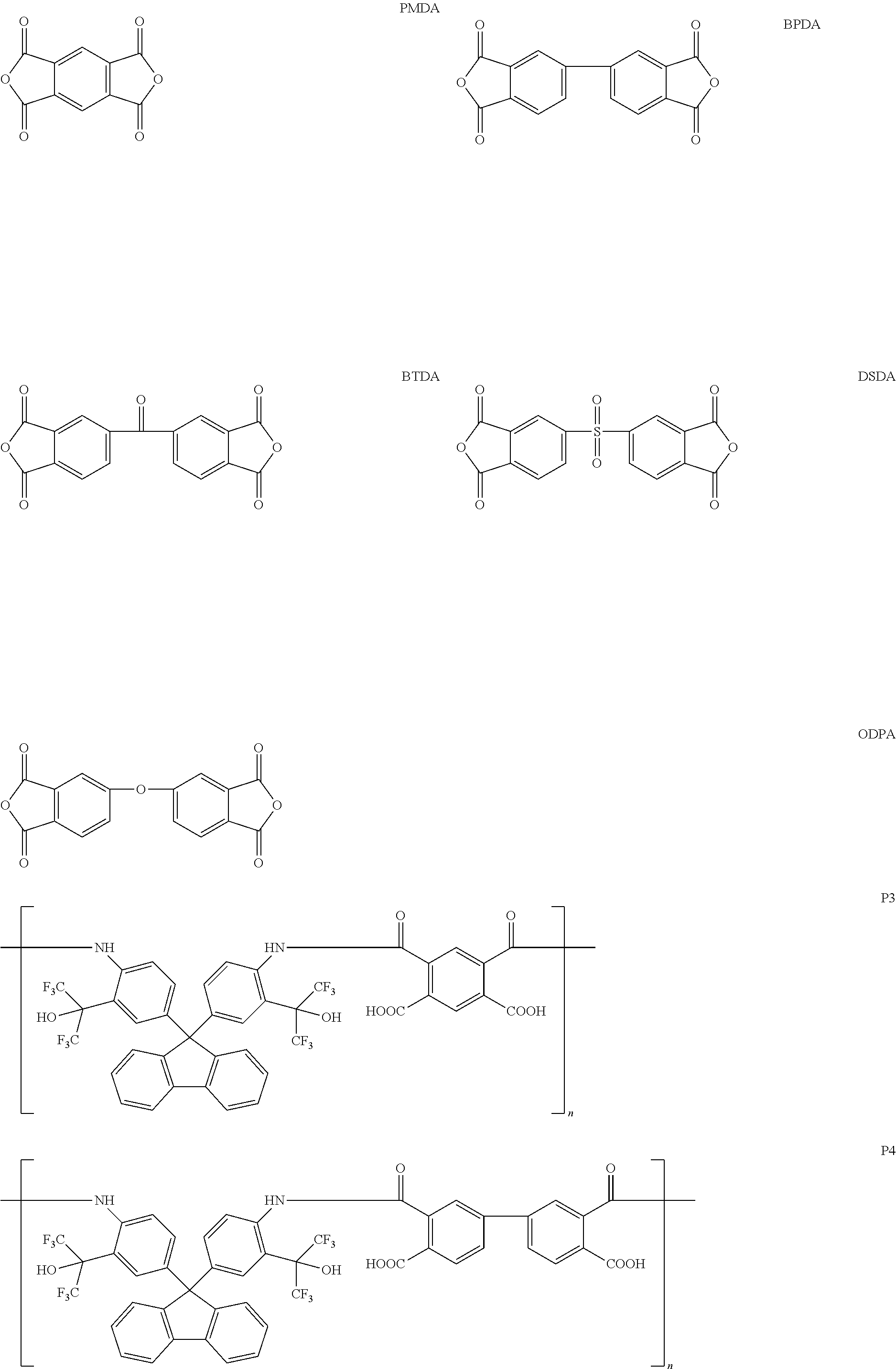

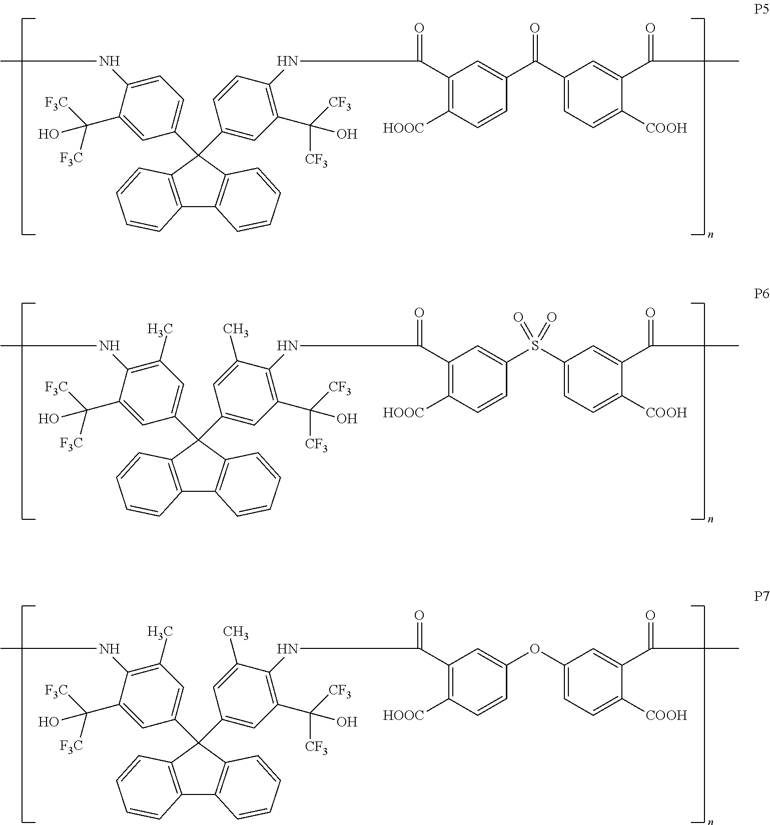

The synthesis results are indicated in TABLE 1.

TABLE 1

| | | HFIP-containing | Tetracarboxylic | |
|---|---|---|---|---|
| Example | Polymer | fluorenediamine | dianhydride | Mw |
| 9 | P3 | HFIP-FL | PMDA | 35,000 |
| 10 | P4 | HFIP-FL | BPDA | 55,000 |
| 11 | P5 | HFIP-FL | BTDA | 42,000 |
| 12 | P6 | HFIP-MeFL | DSDA | 24,000 |
| 13 | P7 | HFIP-MeFL | ODPA | 22,000 |

### Examples 14 to 17

### Synthesis of Polymers P8 to P11

The following polymers P8 to P11 were each synthesized by polymerization of HFIP-FL or HFIP-MeFL with various dicarboxylic acid dichloride such as TPC, BPDC, 6FDC or 6FBDC in the same manner as in Example 6.

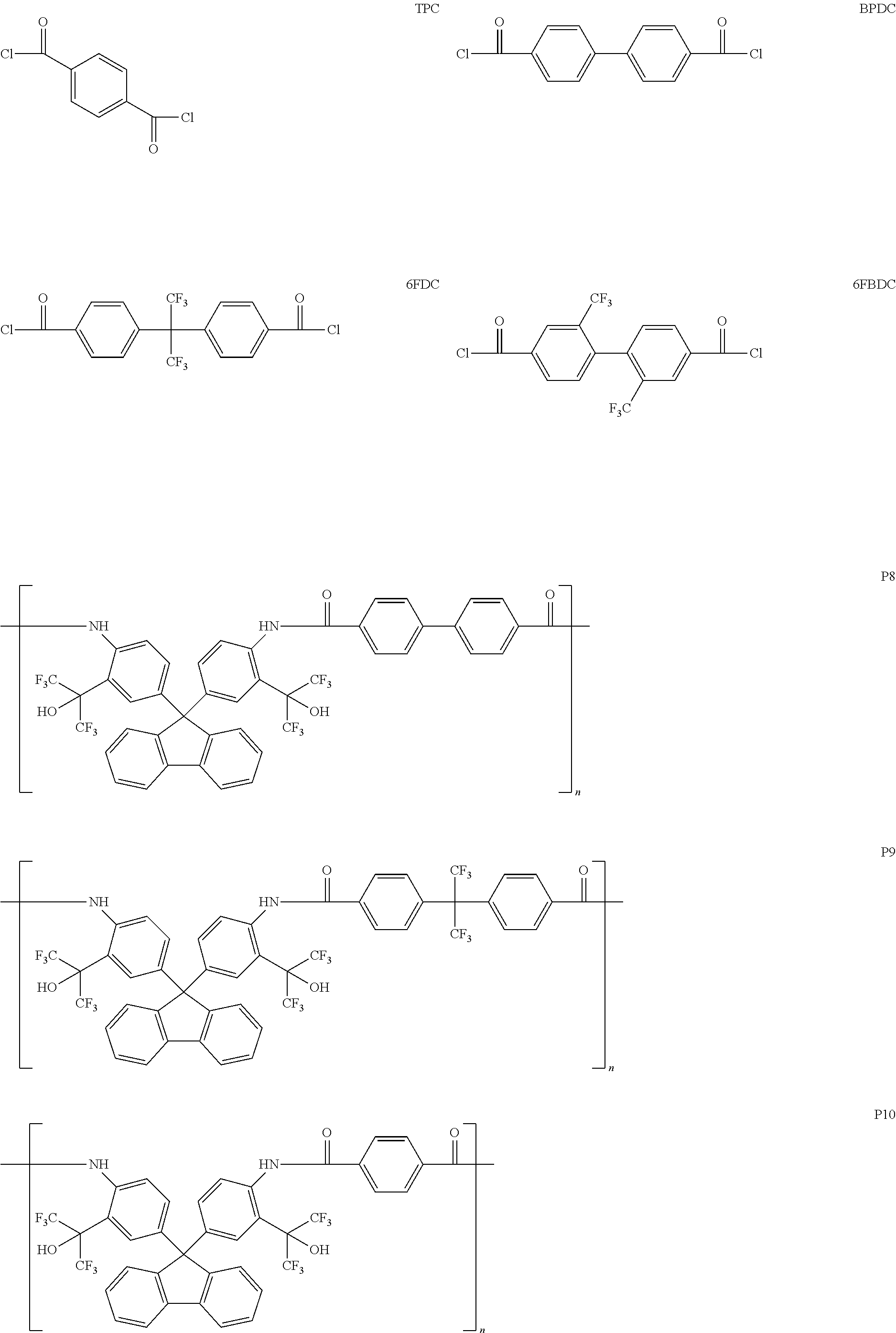

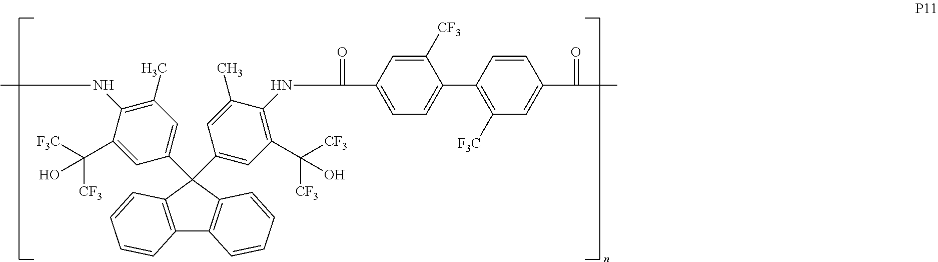

The synthesis results are indicated in TABLE 2.

TABLE 2

| | | HFIP-containing | Dicarboxylic acid | |
|---|---|---|---|---|
| Example | Polymer | fluorenediamine | dichloride | Mw |
| 14 | P8 | HFIP-FL | BPDC | 31,000 |
| 15 | P9 | HFIP-FL | 6FDC | 28,000 |
| 16 | P10 | HFIP-FL | TPC | 41,000 |
| 17 | P11 | HFIP-MeFL | 6FBDC | 30,000 |

### Examples 18 to 22

### Synthesis and Solvent Solubility Test of Polymers P3-1 to P7-1

The following polymers P3-1 to P7-1 were synthesized from the respective polyamic acids, that is, polymers P3 to P7, in the same manner as in Example 4.

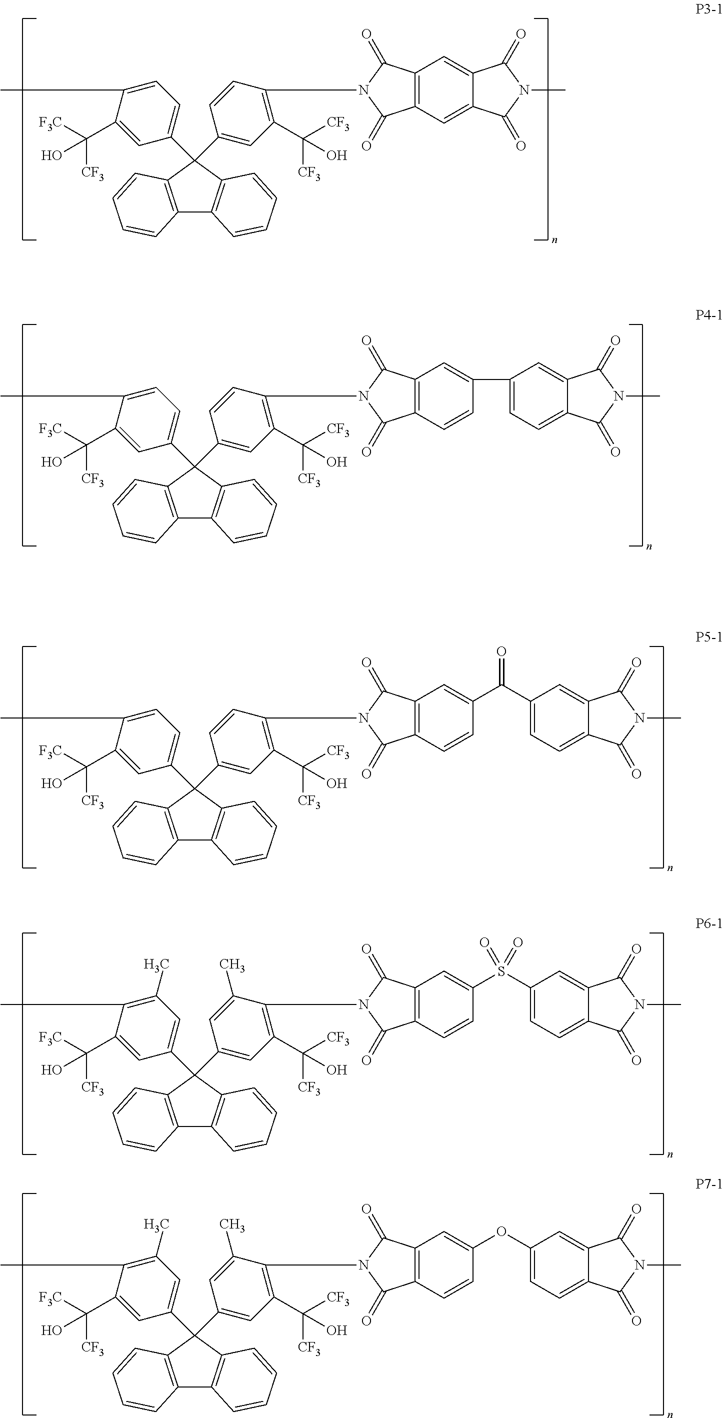

The synthesis results are indicated in TABLE 3. Each of the polymers P3-1 to P7-1 was soluble in tetrahydrofuran and in N,N-dimethylacetamide.

TABLE 3

| Example | Polymer | Mw |
|---|---|---|
| 18 | P3-1 | 28,000 |
| 19 | P4-1 | 49,000 |
| 20 | P5-1 | 38,000 |
| 21 | P6-1 | 22,000 |
| 22 | P7-1 | 21,000 |

### Examples 23 to 26

### Synthesis and Solvent Solubility Test of Polymers P8-1 to P11-1

The following polymers P8-1 to P11-1 were synthesized by dehydration ring-closing reaction of the respective HFIP-containing polyamides, that is, polymers P8 to P11, in the same manner as in Example 7.

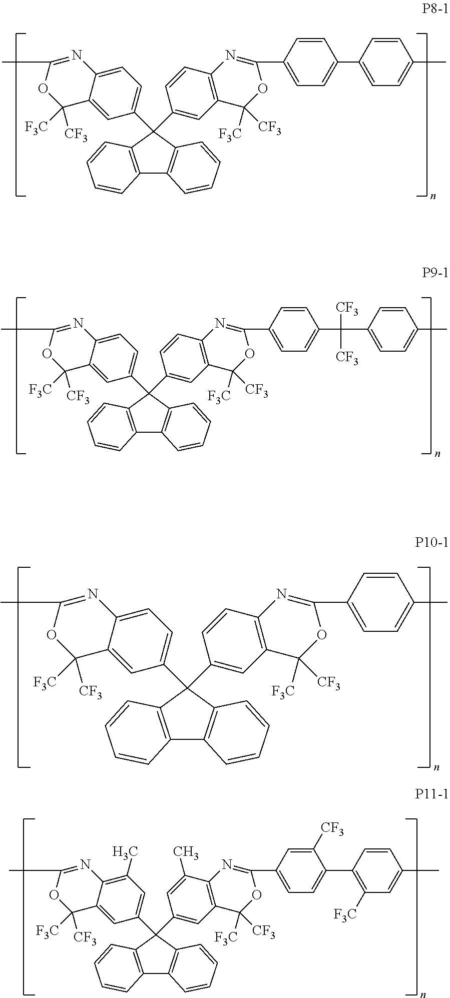

The synthesis results are indicated in TABLE 4. Each of the polymers P8-1 to P11-1 was soluble in N-methylpyrrolidone.

TABLE 4

| Example | Polymer | Mw |
|---|---|---|
| 23 | P8-1 | 26,000 |
| 24 | P9-1 | 24,000 |
| 25 | P10-1 | 35,000 |
| 26 | P11-1 | 28,000 |

### Examples 27 to 35

### Film Formation of Polymers P3-1 to P11-1

Coating films of the polymers P3-1 to P7-1 were formed on glass substrates by spin coating in the same manner as in Example 5. Coating films of the polymers P8-1 to P11-1 were formed on glass substrates by spin coating in the same manner as in Example 8. The film formation results are indicated in TABLE 5.

TABLE 5

| Example | Polymer | Film thickness | Appearance |
|---|---|---|---|
| 27 | P3-1 | 15 μm | No separation and crack |
| 28 | P4-1 | 22 μm | No separation and crack |
| 29 | P5-1 | 19 μm | No separation and crack |
| 30 | P6-1 | 28 μm | No separation and crack |
| 31 | P7-1 | 30 μm | No separation and crack |
| 32 | P8-1 | 16 μm | No separation and crack |
| 33 | P9-1 | 13 μm | No separation and crack |
| 34 | P10-1 | 17 μm | No separation and crack |
| 35 | P11-1 | 20 μm | No separation and crack |

### INDUSTRIAL APPLICABILITY

The coating film of the HFIP-containing polymer compound according to the present invention is useful as coatings of flat panel displays, protective films of electronic circuit boards, protective films of semiconductor devices and the like.

1. A fluorine-containing fluorenediamine of the formula (2): 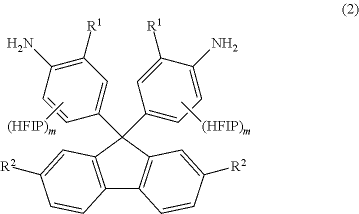
where R¹ represents one kind of substituent group selected from the group consisting of a hydrogen atom, a fluorine atom, a chlorine atom, a bromine atom, a C₁-C₄ alkyl group, a C₁-C₄ fluoroalkyl group in which any number of hydrogen atoms can be substituted by a fluorine atom, a phenyl group, a methoxy group and a nitro group; R² represents one kind of substituent group selected from the group consisting of a hydrogen atom, a fluorine atom, a chlorine atom, a bromine atom, an iodine atom, a phenyl group, a naphthyl group, a biphenyl group, a sulfonic acid group, a —C≡C—C(CH₃)₂OH group, a —C≡C—C₆H₅ group and a —C≡C—Si(CH₃)₃ group; HFIP represents a —C(CF₃)₂OH group; and m and n each independently represent an integer of 0 to 2 and satisfy a condition of 1≦m+n≦4.

2. The fluorine-containing fluorenediamine according to claim 1, wherein the fluorine-containing fluorenediamine is of the formula (3-1): 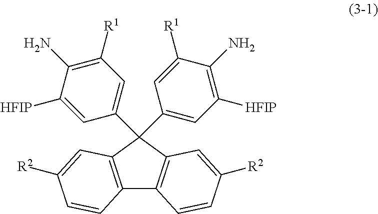
where R¹, R² and HFIP have the same meanings as those in the formula (2).

3. The fluorine-containing fluorenediamine according to claim 2, wherein the fluorine-containing fluorenediamine is of the formula (2B): 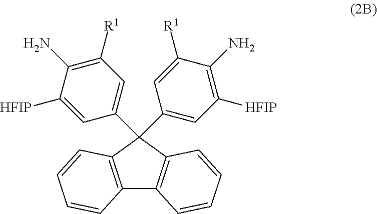
where R¹ and HFIP have the same meanings as those in the formula (2).

4. A polymer compound obtained by reaction of the fluorine-containing fluorenediamine according to claim 1 with a dicarboxylic acid or dicarboxylic acid derivative of the formula (10) or (11) or a tetracarboxylic acid dianhydride of the formula (14) 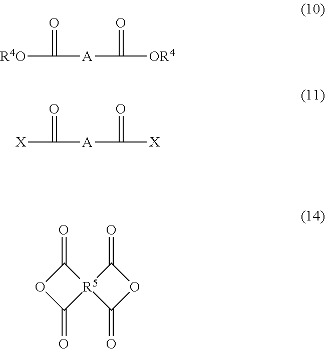
where A represents a divalent organic group having one or more kinds selected from the group consisting of alicyclic, aromatic and alkylene groups and may contain an oxygen atom, a sulfur atom or a nitrogen atom; any number of hydrogen atoms in A may be substituted by an alkyl group, a fluorine atom, a chlorine atom, a fluoroalkyl group, a carboxyl group, a hydroxy group or a cyano group; R⁴ each independently represents a hydrogen atom, a C₁-C₁₀ alkyl group or a benzyl group; X represents a fluorine atom, a chlorine atom, a bromine atom or an iodine atom; R⁵ represents a tetravalent organic group having one or more kinds selected from the group consisting of alicyclic, aromatic and alkylene groups and may contain a fluorine atom, a chlorine atom, an oxygen atom, a sulfur atom or a nitrogen atom; and any number of hydrogen atoms in R⁵ may be substituted by an alkyl group, a fluoroalkyl group, a carboxyl group, a hydroxy group or a cyano group.

5. A polymer compound comprising at least a repeating unit of the formula (6): 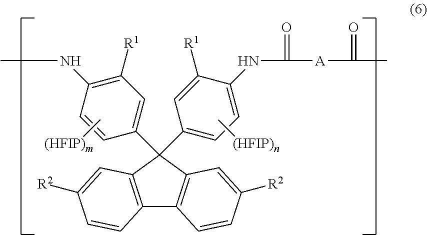
where R¹ represents one kind of substituent group selected from the group consisting of a hydrogen atom, a fluorine atom, a chlorine atom, a bromine atom, a C₁-C₄ alkyl group, a C₁-C₄ fluoroalkyl group in which any number of hydrogen atoms can be substituted by a fluorine atom, a phenyl group, a methoxy group and a nitro group; R² represents one kind of substituent group selected from the group consisting of a hydrogen atom, a fluorine atom, a chlorine atom, a bromine atom, an iodine atom, a phenyl group, a naphthyl group, a biphenyl group, a sulfonic acid group, a —C≡C—C(CH₃)₂OH group, a —C≡C—C₆H₅ group and a —C≡C—Si(CH₃)₃ group; A represents a divalent organic group having one or more kinds selected from the group consisting of alicyclic, aromatic and alkylene groups and may contain an oxygen atom, a sulfur atom or a nitrogen atom; any number of hydrogen atoms in A may be substituted by an alkyl group, a fluorine atom, a chlorine atom, a fluoroalkyl group, a carboxyl group, a hydroxy group or a cyano group; HFIP represents a —C(CF₃)₂OH group; and m and n each independently represent an integer of 0 to 2 and satisfy a condition of 1≦m+n≦4.

6. The polymer compound according to claim 5, wherein the repeating unit is of the formula (19): 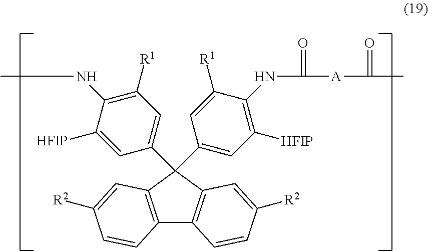
where R¹, R², A and HFIP have the same meanings as those in the formula (6).

7. The polymer compound according to claim 6, wherein the repeating unit is of the formula (6A): 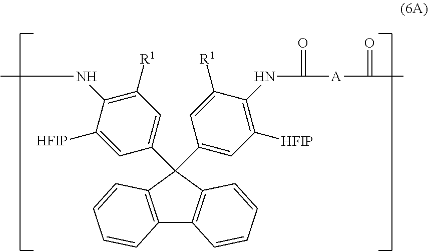
where R¹, A and HFIP have the same meanings as those in the formula (6).

8. A polymer compound comprising at least a repeating unit of the formula (9): 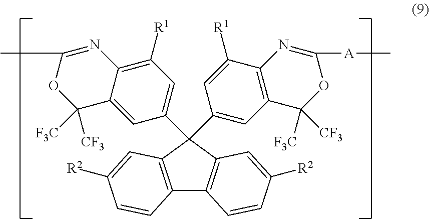
where R¹ represents one kind of substituent group selected from the group consisting of a hydrogen atom, a fluorine atom, a chlorine atom, a bromine atom, a C₁-C₄ alkyl group, a C₁-C₄ fluoroalkyl group in which any number of hydrogen atoms can be substituted by a fluorine atom, a phenyl group, a methoxy group and a nitro group; R² represents one kind of substituent group selected from the group consisting of a hydrogen atom, a fluorine atom, a chlorine atom, a bromine atom, an iodine atom, a phenyl group, a naphthyl group, a biphenyl group, a sulfonic acid group, a —C≡C—C(CH₃)₂OH group, a —C≡C—C₆H₅ group and a —C≡C—Si(CH₃)₃ group; A represents a divalent organic group having one or more kinds selected from the group consisting of alicyclic, aromatic and alkylene groups and may contain an oxygen atom, a sulfur atom or a nitrogen atom; and any number of hydrogen atoms in A may be substituted by an alkyl group, a fluorine atom, a chlorine atom, a fluoroalkyl group, a carboxyl group, a hydroxy group or a cyano group.

9. The polymer compound according to claim 8, wherein the repeating unit is of the formula (9B): 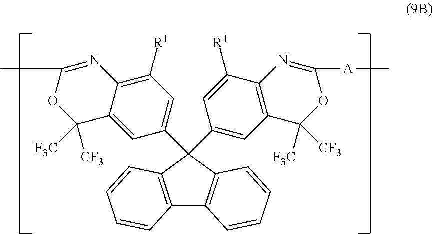
where R¹ and A have the same meanings as those in the formula (9).

10. A polymer compound comprising at least a repeating unit of the formula (7): 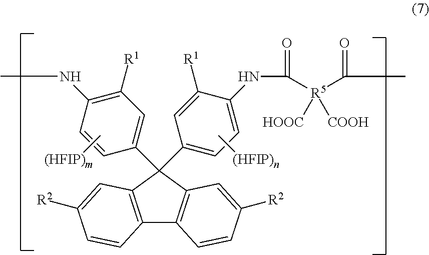
where R¹ represents one kind of substituent group selected from the group consisting of a hydrogen atom, a fluorine atom, a chlorine atom, a bromine atom, a C₁-C₄ alkyl group, a C₁-C₄ fluoroalkyl group in which any number of hydrogen atoms can be substituted by a fluorine atom, a phenyl group, a methoxy group and a nitro group; R² represents one kind of substituent group selected from the group consisting of a hydrogen atom, a fluorine atom, a chlorine atom, a bromine atom, an iodine atom, a phenyl group, a naphthyl group, a biphenyl group, a sulfonic acid group, a —C≡C—C(CH₃)₂OH group, a —C≡C—C₆H₅ group and a —C≡C—Si(CH₃)₃ group; R⁵ represents a tetravalent organic group having one or more kinds selected from the group consisting of alicyclic, aromatic and alkylene groups and may contain a fluorine atom, a chlorine atom, an oxygen atom, a sulfur atom or a nitrogen atom; and any number of hydrogen atoms in R⁵ may be substituted by an alkyl group, a fluoroalkyl group, a carboxyl group, a hydroxy group or a cyano group; HFIP represents a —C(CF₃)₂OH group; and m and n each independently represent an integer of 0 to 2 and satisfy a condition of 1≦m+n≦4.

11. The polymer compound according to claim 10, wherein the repeating unit is of the formula (7A): 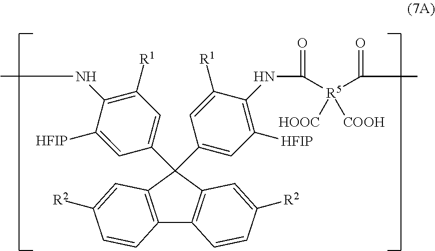
where R¹, R², R⁵ and HFIP have the same meanings as those in the formula (7).

12. The polymer compound according to claim 11, wherein the repeating unit is of the formula (7B): 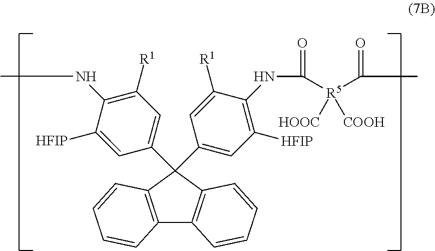
where R¹, R⁵ and HFIP have the same meanings as those in the formula (7).

13. A polymer compound comprising at least a repeating unit of the formula (8): 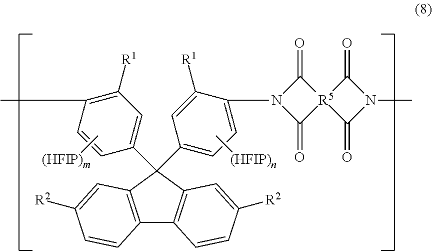
where R¹ represents one kind of substituent group selected from the group consisting of a hydrogen atom, a fluorine atom, a chlorine atom, a bromine atom, a C₁-C₄ alkyl group, a C₁-C₄ fluoroalkyl group in which any number of hydrogen atoms can be substituted by a fluorine atom, a phenyl group, a methoxy group and a nitro group; R² represents one kind of substituent group selected from the group consisting of a hydrogen atom, a fluorine atom, a chlorine atom, a bromine atom, an iodine atom, a phenyl group, a naphthyl group, a biphenyl group, a sulfonic acid group, a —C≡C—C(CH₃)₂OH group, a —C≡C—C₆H₅ group and a —C≡C—Si(CH₃)₃ group; R⁵ represents a tetravalent organic group having one or more kinds selected from the group consisting of alicyclic, aromatic and alkylene groups and may contain a fluorine atom, a chlorine atom, an oxygen atom, a sulfur atom or a nitrogen atom; and any number of hydrogen atoms in R⁵ may be substituted by an alkyl group, a fluoroalkyl group, a carboxyl group, a hydroxy group or a cyano group; HFIP represents a —C(CF₃)₂OH group; and m and n each independently represent an integer of 0 to 2 and satisfy a condition of 1≦m+n≦4.

14. The polymer compound according to claim 13, wherein the repeating unit is of the formula (8A): 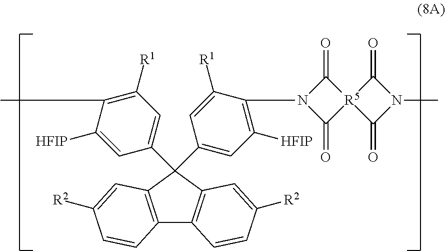
where R¹, R², R⁵ and HFIP have the same meanings as those in the formula (8).

15. The polymer compound according to claim 14, wherein the repeating unit is of the formula (8B): 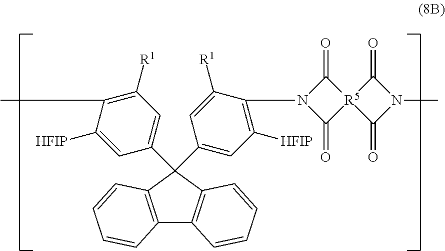
where R¹, R⁵ and HFIP have the same meanings as those in the formula (8).

16. The polymer compound according to claim 5, wherein the divalent group A is one or more kinds selected from the group consisting of groups of the formulas (25) to (29): 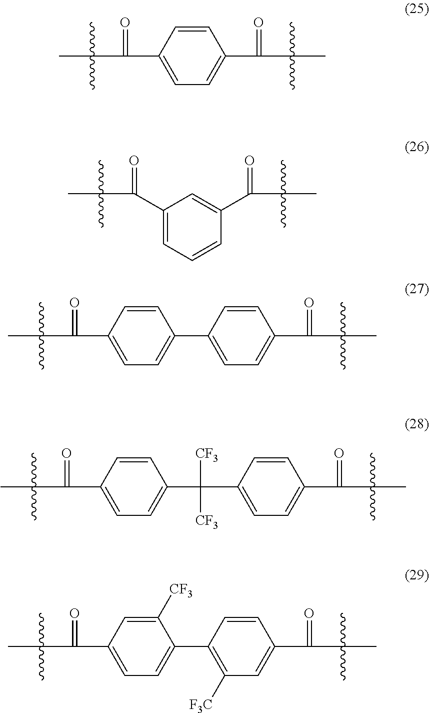 [SMILES: *C(=O)c1ccc(C(*)=O)cc1 ; *C(=O)c1cccc(C(*)=O)c1 ; *C(=O)c1ccc(-c2ccc(C(*)=O)cc2)cc1 ; *C(=O)c1ccc(C(c2ccc(C(*)=O)cc2)(C(F)(F)F)C(F)(F)F)cc1 ; *C(=O)c1ccc(-c2ccc(C(*)=O)cc2C(F)(F)F)c(C(F)(F)F)c1]
wherein each line intersecting a wavy line represents a bonding position.

17. The polymer compound according to claim 10, wherein R⁵ is one or more kinds selected from the group consisting of groups of the formulas (30) to (35): 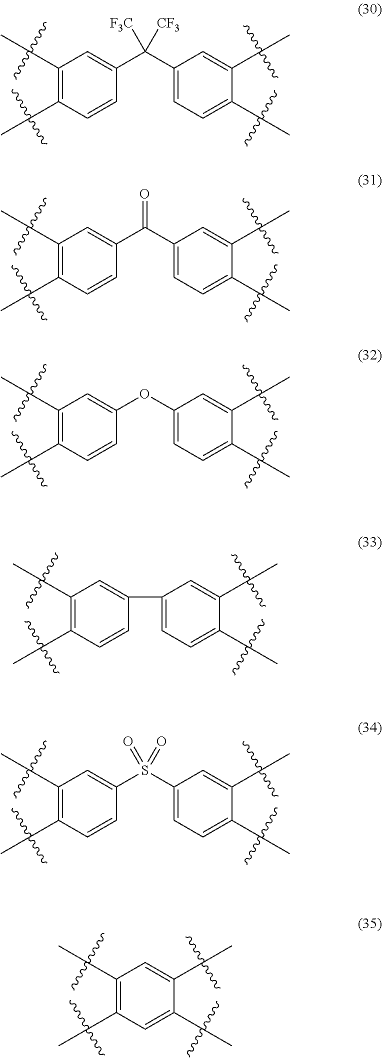 [SMILES: *c1ccc(C(c2ccc(*)c(*)c2)(C(F)(F)F)C(F)(F)F)cc1* ; *c1ccc(C(=O)c2ccc(*)c(*)c2)cc1* ; *c1ccc(Oc2ccc(*)c(*)c2)cc1* ; *c1ccc(-c2ccc(*)c(*)c2)cc1* ; *c1ccc(S(=O)(=O)c2ccc(*)c(*)c2)cc1* ; *c1cc(*)c(*)cc1*]
wherein each line intersecting a wavy line represents a bonding position.

18. The polymer compound according to claim 8, wherein the divalent group A is one or more kinds selected from the group consisting of groups of the formulas (25) to (29): 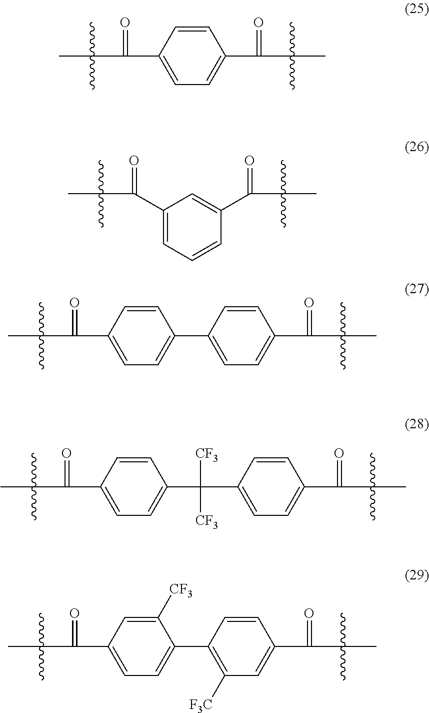 [SMILES: *C(=O)c1ccc(C(*)=O)cc1 ; *C(=O)c1cccc(C(*)=O)c1 ; *C(=O)c1ccc(-c2ccc(C(*)=O)cc2)cc1 ; *C(=O)c1ccc(C(c2ccc(C(*)=O)cc2)(C(F)(F)F)C(F)(F)F)cc1 ; *C(=O)c1ccc(-c2ccc(C(*)=O)cc2C(F)(F)F)c(C(F)(F)F)c1]
wherein each line intersecting a wavy line represents a bonding position.

19. The polymer compound according to claim 13, wherein R⁵ is one or more kinds selected from the group consisting of groups of the formulas (30) to (35): 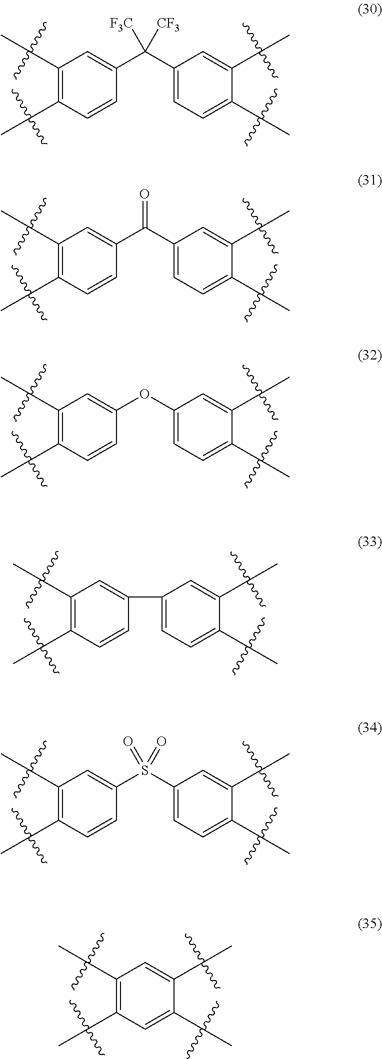 [SMILES: *c1ccc(C(c2ccc(*)c(*)c2)(C(F)(F)F)C(F)(F)F)cc1* ; *c1ccc(C(=O)c2ccc(*)c(*)c2)cc1* ; *c1ccc(Oc2ccc(*)c(*)c2)cc1* ; *c1ccc(-c2ccc(*)c(*)c2)cc1* ; *c1ccc(S(=O)(=O)c2ccc(*)c(*)c2)cc1* ; *c1cc(*)c(*)cc1*]
wherein each line intersecting a wavy line represents a bonding position.
